(19) United States
(12) Patent Application Publication
(10) Pub. No.: US 2018/0080925 A1
(43) Pub. Date: Mar. 22, 2018

(54) RESPIRATION DEVICE FOR ANALYSIS OF A RESPONSE TO SHEAR STRESS AND FOREIGN AGENTS ON CELLS

(71) Applicant: PRESIDENT AND FELLOWS OF HARVARD COLLEGE, Cambridge, MA (US)

(72) Inventors: Kambez Hajipouran Benam, Cambridge, MA (US); Richard Novak, Jamaica Plain, MA (US); Josiah Sliz, Boston, MA (US); Thomas Charles Ferrante, Newton, MA (US); Donald Elliot Ingber, Boston, MA (US); Youngjae Choe, Somerville, MA (US)

(21) Appl. No.: 15/563,140

(22) PCT Filed: Mar. 31, 2016
(86) PCT No.: PCT/US16/25228

§ 371 Date: Sep. 29, 2017

Related U.S. Application Data
(60) Provisional application No. 62/141,560, filed on Apr. 1, 2015.

(51) Int. Cl. G01N 33/50; B01L 3/00; F04B 49/06

(52) U.S. Cl. CPC G01N 33/5064; B01L 3/50273; B01L 3/502723; B01L 3/502761; F04B 49/06; F04B 19/22; B01L 2400/049; B01L 2400/0478; B01L 2300/0861; B01L 2200/025; B01L 2200/0689; B01L 2200/14

(57) ABSTRACT

A microfluidic system for determining a response of cells comprises one or more fluid pumps. The one or more fluid pumps move a fluid across cells within a microfluidic device. The microfluidic device includes a microchannel at least partially defined by a surface having cells adhered thereto, a first port at one end of the microchannel, and a second port at an opposing end of the microchannel. The one or more fluid pumps move the fluid across the cells in a first direction toward the second port and then move the fluid across the cells in a second direction toward the first port.

### CROSS-REFERENCE TO RELATED APPLICATIONS

This application claims the benefit of U.S. Provisional Application No. 62/141,560, entitled, “RESPIRATION DEVICE FOR ANALYSIS OF A RESPONSE TO SHEAR STRESS AND FOREIGN AGENTS ON CELLS,” filed Apr. 1, 2015, the disclosure of which is incorporated by reference herein in its entirety, including drawings.

### GOVERNMENT SUPPORT

This invention was made with government support under grant number W911NF-12-2-0036 awarded by Defense Advanced Research Projects Agency (DARPA). The government has certain rights in the invention.

### TECHNICAL FIELD

The present invention relates to cell culture systems and fluidic systems. More specifically, the invention relates to a system for testing responses of cell culture systems in microfluidic devices to shear stress and various agents introduced into the fluidic systems.

### BACKGROUND

Microfluidic and/or mesofluidic devices (hereinafter referred to as microfluidic devices) allow for various types of experimentation on various types of cells contained within the devices. The experimentation often requires the flow of fluid across the cells, which mimics the flow of fluid in vivo. With respect to cells associated with a respiratory function (e.g., lung cells), the flow of fluid is bi-directional. During normal respiratory function, cells experience a flow of fluid, such as air, in a first direction as the air is breathed in, followed by a flow of fluid in a second, opposite direction as the air is breathed out. The amount of fluid that passes through the microfluidic devices range from microliters to milliliters.

The bi-directional flow of fluid through microfluidic devices is difficult to achieve with existing equipment. Moreover, existing equipment cannot provide such bi-directional flow at controllable flow rates at the small microliter and/or milliliter volumes required to mimic flow in vivo. By way of example, certain small animal ventilators exist for assisting in breathing. Yet, such ventilators are unable to provide microliters of fluid flow, such as within the range of 10-30 μl. Such ventilators also are unable to deliver exact volumes of fluid because of high oscillations and discrepancies between volumes as set on the instrument and volumes actually delivered to the microfluidic devices. Such ventilators typically also have pre-set fluid flow rates that cannot be modified or deviated from, are large and, therefore, require a large space within the testing environment, and are not designed for open-ended microfluidic devices.

Moreover, there are no suitable devices that currently exist for mimicking the effects of the respiration of one or more agents within a fluid in microfluidic devices. For example, setups exist that generate smoke for exposure to differentiated airway epithelial cells in vitro. However, these systems have been developed for cells grown on plates, transwell inserts, or other static cell culture systems—not microfluidic devices—and the systems only introduce smoke as a single puff on top of the cells. Such systems are incapable of delivering smoke by mimicking breathing movements across epithelial cells in vivo.

The below-described devices, methods, and systems solve many of the problems associated with the current art by providing a fluid pump that can accurately and precisely control the fluid flow across a microfluidic device. The below-described devices, methods, and systems also provide a way for introducing one or more agents into the fluid to investigate the effects of the agents on the microfluidic devices, particularly in a way that closely mimics the conditions in vivo. Further, the below-described devices, methods, and systems also allow for a controlled burning of one or more smokeable products to introduce the smoke as the one or more agents into the fluid flowed across the microfluidic device.

### SUMMARY

According to aspects of the present disclosure, a system is presented that cyclically breathes air and cigarette smoke into and out of a microfluidic device lined with living cells.

According to additional aspects of the present disclosure, a mechano-electrical instrument (e.g., a fluid pump) is disclosed that introduces air into and out of a microfluidic device at volumes, rates, and oscillating patterns that are physiologically relevant to in vivo conditions.

According to additional aspects of the present disclosure, a smoking-generating apparatus is disclosed for introducing smoke (e.g., cigarette smoke) into a fluid that is drawn into and out of microfluidic devices.

According to additional aspects of the present disclosure, a microfluidic device is disclosed for determining a response of cells. The microfluidic device includes a body and a porous membrane. The body at least partially defines a first microchannel and a second microchannel. The first microchannel is configured for a bi-directional flow of fluid through the microfluidic device, and the second microchannel is configured for a flow of fluid through the microfluidic device. The porous membrane at least partially defines the first microchannel and the second microchannel and includes the cells on at least a portion of the porous membrane that partially defines the first microchannel.

According to a further aspect of the present disclosure, a microfluidic system for determining a response of cells comprises one or more fluid pumps. The one or more fluid pumps move a fluid across cells within a microfluidic device. The microfluidic device includes a microchannel at least partially defined by a surface having cells adhered thereto, a first port at one end of the microchannel, and a second port at an opposing end of the microchannel. The one or more fluid pumps move the fluid across the cells in a first direction toward the second port and then move the fluid across the cells in a second direction toward the first port.

According to another aspect of the present invention, a fluid pump for producing bi-directional movement of a fluid within one or more microfluidic devices is disclosed. The fluid pump comprises at least one syringe, a traveling nut, and a motor. The at least one syringe comprises a movable plunger and has an end with a port in fluid communication with at least one of the microfluidic devices. The traveling nut is coupled to the at least one syringe. The motor is coupled to the traveling nut for moving the traveling nut in a first direction, which actuates the syringe and results in the fluid moving into the port. The motor coupled to the traveling nut is also for moving the traveling nut in a second direction, which actuates the syringe and results in the fluid moving out of the port.

According to another aspect of the present invention, an alternative fluid pump for producing bi-directional movement of a fluid within one or more microfluidic devices comprises a plurality of syringes. Each of the syringes comprises a plunger supported within a barrel and an end with a port in fluid communication with at least one of the one or more microfluidic devices. The fluid pump further comprises a first plate and a second plate. The first plate is fixed to the plurality of barrels at ends of the plurality of syringes. The second plate is fixed to the plurality of plungers at opposing ends of the plurality of syringes. The fluid pump further comprises a lead screw extending between the first plate and the second plate and a motor coupled to the lead screw for rotating the lead screw in a first rotational direction and a second rotational direction. The fluid pump further comprises a traveling nut coupled to one of the first plate and the second plate and rotatable about the lead screw. Operation of the motor causes the one of the first plate and the second plate to translate about the lead screw when the lead screw rotates in the first rotational direction and the second rotational direction. The rotation of the lead screw causes the plurality of syringes to draw in and push out the fluid to bi-directionally move the fluid within the microfluidic devices.

According to another aspect of the present invention, an apparatus for introducing smoke into a fluid for delivery to a microfluidic device comprises a plate that includes one or more indents on a first side. Each indent is configured to couple a smokeable product to the plate. The one or more smokeable products extend from the plate in a first direction. The apparatus further comprises a seal piece configured to selectively engage with the plate to create a seal on a second side of the plate, opposing one of the one or more indents. The apparatus further comprises a tube coupled to and extending from the seal piece in a second direction, opposite to the first direction. The tube is in fluid communication with the microfluidic device for supplying the fluid and the smoke to the microfluidic device.

According to a further aspect of the present disclosure, a method of bi-directionally flowing fluid is disclosed. The method includes providing (i) a microfluidic device comprising a body that at least partially defines a microchannel and (ii) a fluid. The method further includes introducing a portion of said fluid into said microchannel so as to cause said fluid to move in a first direction. The method further includes causing said fluid to move in a second direction, thereby bi-directionally flowing fluid.

According to an additional aspect of the present disclosure, a method for introducing smoke to a microfluidic device is disclosed. The method includes providing a microfluidic device in fluid communication with a smoking device. The smoking device comprises a receptacle with a smokeable product coupled thereto. The smokeable product is capable of generating smoke when ignited. The smoking device further comprises a tube in fluid communication with said microfluidic device for supplying the smoke to the microfluidic device. The microfluidic device also comprises a body that at least partially defines a microchannel, with the microchannel including cells. The method further includes igniting said smokeable product under conditions that generate smoke. The method further includes delivering said smoke to said microchannel under conditions such that said smoke contacts said cells.

These and other capabilities of the inventions, along with the inventions themselves, will be more fully understood after a review of the following figures, detailed description, and claims.

### BRIEF DESCRIPTION OF THE DRAWINGS

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

FIG. 1 illustrates a microfluidic device with a membrane region having cells thereon, in accord with some aspects of the present concepts.

FIG. 2 is a cross-section of the microfluidic device taken along line 2-2 of FIG. 1, illustrating the membrane separating a first microchannel and a second microchannel, in accord with some aspects of the present concepts.

FIG. 3A is a perspective view of a fluid pump, in accord with some aspects of the present concepts.

FIG. 3B is a detailed view of a motor of the fluid pump, in accord with some aspects of the present concepts.

FIG. 3C is a detailed view of a coupler and lead screw of the fluid pump, in accord with some aspects of the present concepts.

FIG. 3D is a detailed view of the lead screw coupled to an end plate of the fluid pump, in accord with some aspects of the present concepts.

FIG. 3E is a detailed view of a linear bearing on a guide rail of the fluid pump, in accord with some aspects of the present concepts.

FIG. 3F is a detailed view of a traveling nut of the fluid pump, in accord with some aspects of the present concepts.

FIG. 3G is a detailed view of syringes of the fluid pump, in accord with some aspects of the present concepts.

FIG. 3H is a detailed view of a limit switch of the fluid pump, in accord with some aspects of the present concepts.

FIG. 3I is a diagram of a controller of the fluid pump, in accord with some aspects of the present concepts.

FIG. 4 is a perspective view of an alternative configuration of the fluid pump of FIG. 3A, in accord with some aspects of the present concepts.

FIG. 5 is a layout of a system for introducing bi-directional fluid flow through a microfluidic device, in accord with some aspects of the present concepts.

FIG. 6A is a perspective view of a smoking apparatus that can be used within the system of FIG. 5, in accord with some aspects of the present concepts.

FIG. 6B is a detailed view of a rotatable wheel and motor of the smoking apparatus of FIG. 6A, in accord with some aspects of the present concepts.

FIG. 6C is a cross-section view along the line 6C of a sealing member engaging with a rotatable wheel of the smoking apparatus of FIG. 6A, in accord with some aspects of the present concepts.

FIG. 6D is a cross-section view along the line 6D-6D of the sealing member engaging with the rotatable when of the smoking apparatus of FIG. 6A, in accord with some aspects of the present concepts.

FIG. 6E is a detailed view of the mechanism for actuating the sealing member of the smoking apparatus of FIG. 6A, in accord with some aspects of the present concepts.

FIG. 6F is a perspective view of the rotatable wheel with smokeable products of the smoking apparatus of FIG. 6A, in accord with some aspects of the present concepts.

FIG. 6G is a perspective view of an ignition system of the smoking apparatus of FIG. 6A, in accord with some aspects of the present concepts.

FIG. 6H is a perspective view of a cover of the smoking apparatus of FIG. 6A, in accord with some aspects of the present concepts.

FIG. 7 is a flow diagram of a method of introducing one or more agents into a fluid within the system of FIG. 5, in accord with some aspects of the present concepts.

### DETAILED DESCRIPTION

While the inventions are susceptible of embodiment in many different forms, there is shown in the drawings and will herein be described in detail preferred embodiments of the inventions with the understanding that the present disclosure is to be considered as an exemplification of the principles of the inventions and is not intended to limit the broad aspects of the inventions to the embodiments illustrated.

The functionality of cells and tissue types (and even organs) can be implemented in one or more microfluidic devices or “chips” that enable researchers to study these cells and tissue types outside of the body while mimicking much of the stimuli and environment that the tissue is exposed to in vivo. It can also be desirable to implement these microfluidic devices into interconnected components that can simulate groups of organs or tissue systems. Preferably, the microfluidic devices can be easily inserted and removed from an underlying fluidic system that connects to these devices in order to vary the simulated in vivo conditions and organ systems.

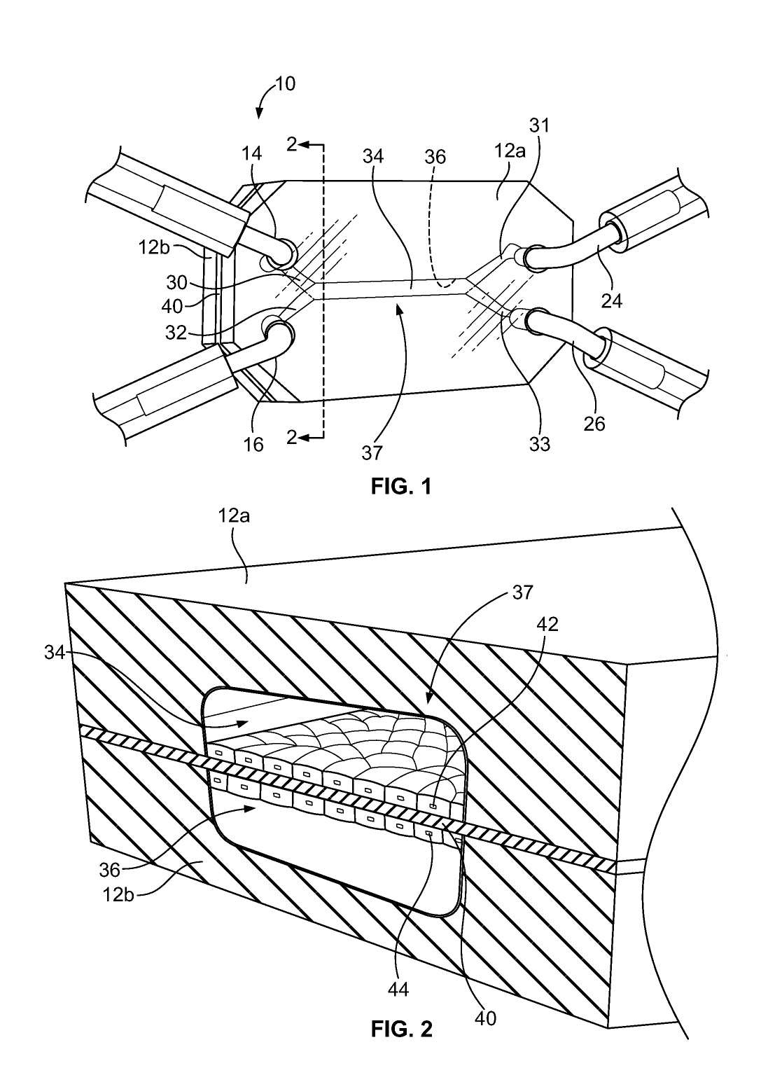

FIGS. 1 and 2 illustrate one type of an organ-on-chip device (“OOC”) 10, in accord to some aspects of the present concepts. Referring to FIG. 1, the OOC 10 includes a body 12 that is typically comprised of an upper body segment 12a and a lower body segment 12b. The upper body segment 12a and the lower body segment 12b are preferably made of a polymeric material, such as polydimethysyloxane (PDMS), poly(methyl methacrylate) (PMMA), polycarbonate, cyclic olefin copolymer (COP), cyclic olefin polymer (COC), polyurethane, styrene-butadiene-styrene (SBS) and/or poly(styrene-ethylene/butylene-styrene) (SEBS) block copolymers, etc. The upper body segment 12a includes a fluid inlet 14 and a fluid outlet 24. A first fluid path for a first fluid includes the fluid inlet 14, a seeding channel 30, an upper microchannel 34, an exit channel 31, and then the fluid outlet 24. The lower body segment 12b includes a fluid inlet 16 and a fluid outlet 26. A second fluid path for a second fluid includes the fluid inlet 16, a seeding channel 32, a lower microchannel 36, an outlet channel 33, and the fluid outlet 26.

Although referred to herein as a fluid inlet 14 and a fluid outlet 24, according to a preferred embodiment, the fluid inlet 14 and the fluid outlet 24 are both an inlet and an outlet, such as in the case of bi-directional flow of fluid through the microchannel 34. By way of example, and without limitation, fluid can flow into the fluid inlet 14 and then flow out of the fluid outlet 24. Subsequently, the fluid can flow back into the fluid outlet 24 and then flow out of the fluid inlet 14. Thus, the terms inlet and outlet are used for purposes of convenience and should not be interpreted as limiting. Alternatively, or in addition, the fluid inlet 16 and the fluid outlet 26 also can be both fluid inlets and fluid outlets, such as in the case of the bi-directional flow of fluid through the microchannel 36.

FIG. 2 shows a cross-section of the OOC 10 of FIG. 1 along the line 2-2. The OOC 10 includes a membrane 40 extends between and separates the upper body segment 12a and the lower body segment 12b. The membrane 40 is preferably an inert, polymeric membrane having uniformly or randomly distributed pores with sizes in the range from about 0.2 μm to about 12 μm in width. The membrane may, for example, be micro-molded, track-etched, laser-machined, fiber-based, or otherwise produced. The thickness of the membrane 40 is generally in the range of about of about 7 μm to about 100 μm. Preferably, the membrane 40 is made of a cured PDMS. The membrane 40 separates the upper microchannel 34 from the lower microchannel 36 in an active region 37, which includes a bilayer of cells in the illustrated embodiment. In particular, a cell layer 42 is adhered to one side of the membrane 40, and a cell layer 44 is adhered to an opposing side of the membrane 40. The cell layer 42 may include the same type of cells as the cell layer 44. Or, the cell layer 42 may include a different type of cells than the cell layer 44. Although a single layer of cells is shown for the cell layer 42 and the cell layer 44, the cell layer 42 and the cell layer 44 may include multiple cell layers. Further, while the illustrated embodiment includes a bilayer of cells on the membrane 40, the membrane 40 may include only a single cell layer of cells disposed on one of its sides.

The OOC 10 is configured to simulate a biological function that typically includes cellular communication between the cell layer 42 and the cell layer 44, as would be experienced in vivo within organs, tissues, cells, etc. Depending on the application, the membrane 40 is designed to have a porosity to permit the migration of cells, particulates, media, proteins, and/or chemicals between the upper microchannel 34 and the lower microchannel 36. The working fluids within the microchannels 34 and 36 may be the same fluid or different fluids. As one example, an OOC 10 simulating a lung may have air as the fluid in one channel and a fluid simulating blood in the other channel. When developing the cell layers 42 and 44 on the membrane 40, the working fluids may be a tissue-culturing fluid.

By way of example, and without limitation, the cell layers 42 and 44 can include human airway epithelial cells (e.g., bronchiolar, bronchial, or tracheal cells). Specifically, the cell layer 42 within the microchannel 34 can include differentiated (pseudostratified ciliated) epithelial cells. The cell layer 44 within the microchannel 36 can include other lung cell types, such as endothelium, macrophages, fibroblasts, and/or other immune cells. Accordingly, the cells can be cells from one or more parts of the airways or respiratory system, including from the lungs (and the various scales of the airway tubes within the lungs, including the alveoli), the windpipe, and the nasal canal. With the cell layer 42 within the microchannel 34, the microchannel 34 resembles an airway lumen of the human respiratory tract.

Although the present disclosure is discussed primarily with respect to an OOC 10, according to some embodiments, other culture systems can be used within the system discussed below (e.g., system 500), or sub-components thereof, without departing from the spirit and scope of the present disclosure. Thus, conventional cell-culture systems can be used in place of the OOC 10 to expose the cells to fluid or fluid mixed with one or more agents. Further, the cells within the OOC 10 and/or the conventional cell-culture systems are not limited to only airway cells. According to some embodiments, other cell types can be used within the OOC 10 or the conventional-cell culture systems. By way of example, and without limitation, other cells can include nasopharynx cells, mouth cells, tongue cells, eye cells, skin cells, vascular cells, cells from the lymphatic system, or any other type of cell for investigating the effects of exposure of the cell to fluid or fluid mixed with one or more agents.

In one embodiment, the active region 37 defined by the microchannels 34 and 36 has a length of less than about 10 cm, a height of less than 1.5 mm, and a width of less than 2000 μm. The OOC 10 preferably includes an optical window that permits viewing of the fluids, media, particulates, etc. as they move across the cell layer 42 and/or the cell layer 44. Various image-gathering techniques, such as spectroscopy and microscopy, can be used to quantify and evaluate the effects of the fluid flow in the microchannels 34 and 36, as well as cellular behavior and cellular communication through the membrane 40. More details on the OOC 10 can be found in, for example, U.S. Pat. No. 8,647,861, which is incorporated by reference in its entirety. Consistent with the disclosure in U.S. Pat. No. 8,647,861, in one preferred embodiment, the membrane 40 is capable of stretching and expanding in one or more planes to simulate the physiological effects of expansion and contraction forces that are commonly experienced by cells.

Although FIGS. 1 and 2 describe a specific type of OOC 10, the aspects of the present concepts can be applied to various other types of devices without departing from the spirit and scope of the present disclosure. By way of example, the present concepts disclosed herein can apply to any microfluidic device (or a plurality of microfluidic devices), and particularly to any type of microfluidic cell-culture device that does not necessarily include a membrane (e.g., membrane 40) that separates one or more microchannels. Thus, the aspects of the present concepts related to experiments associated with microfluidic devices are not restricted to only membrane-based devices.

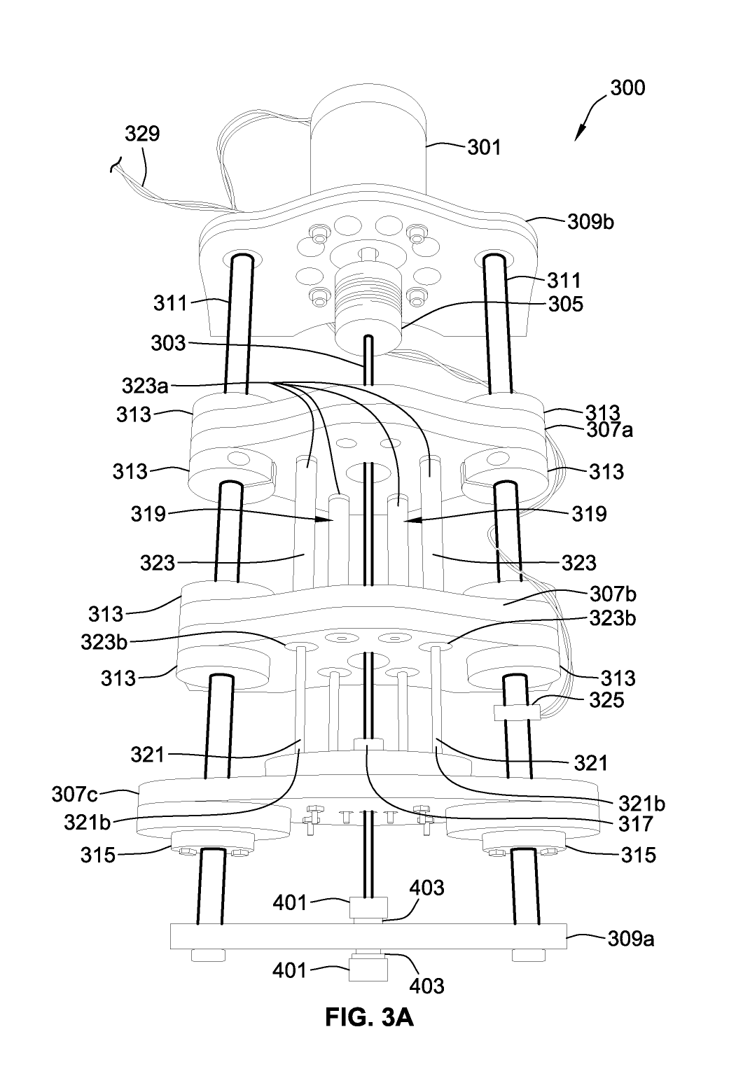

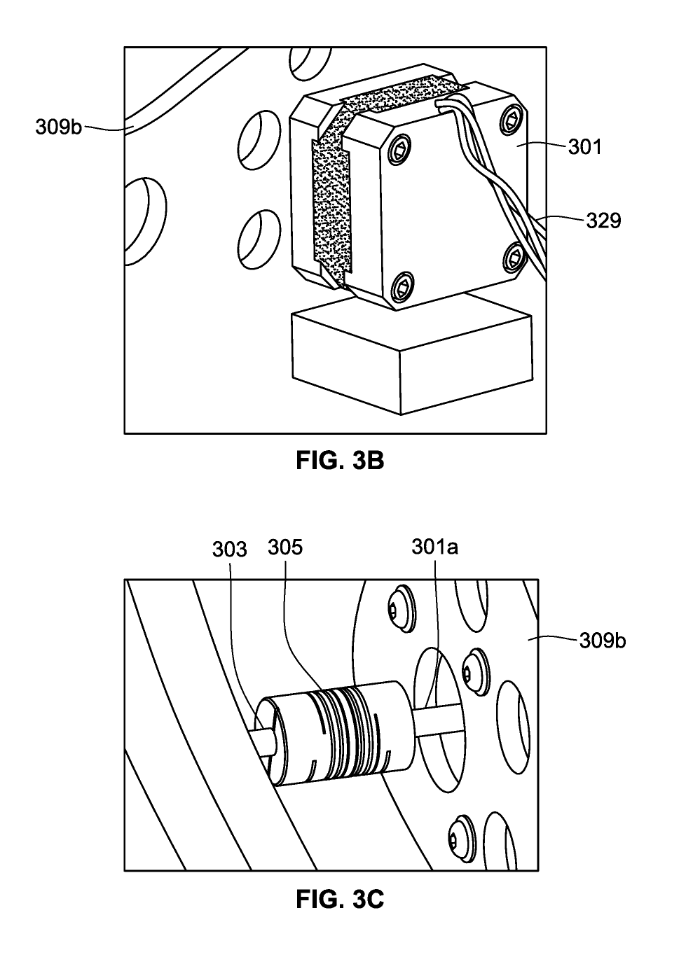

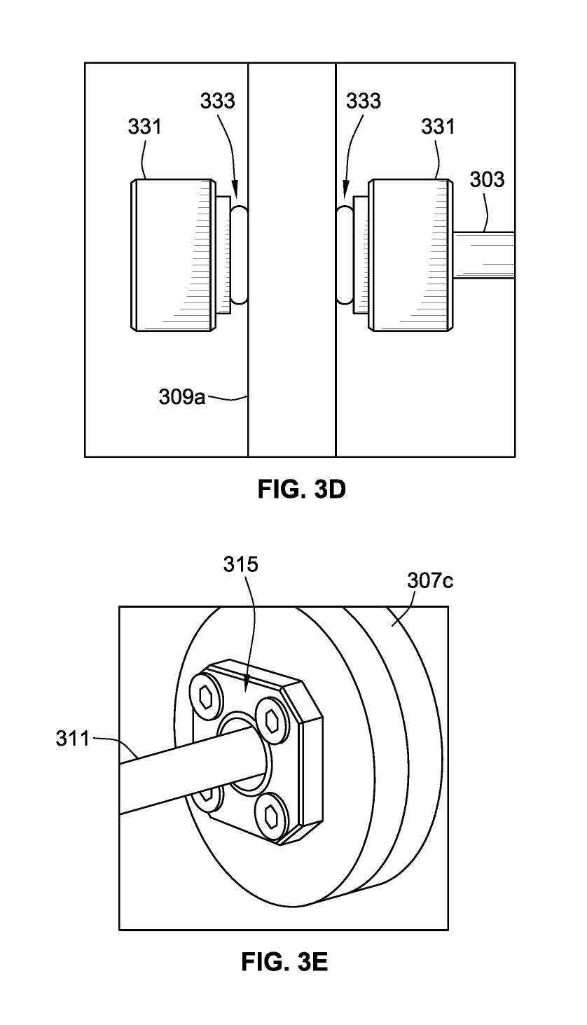

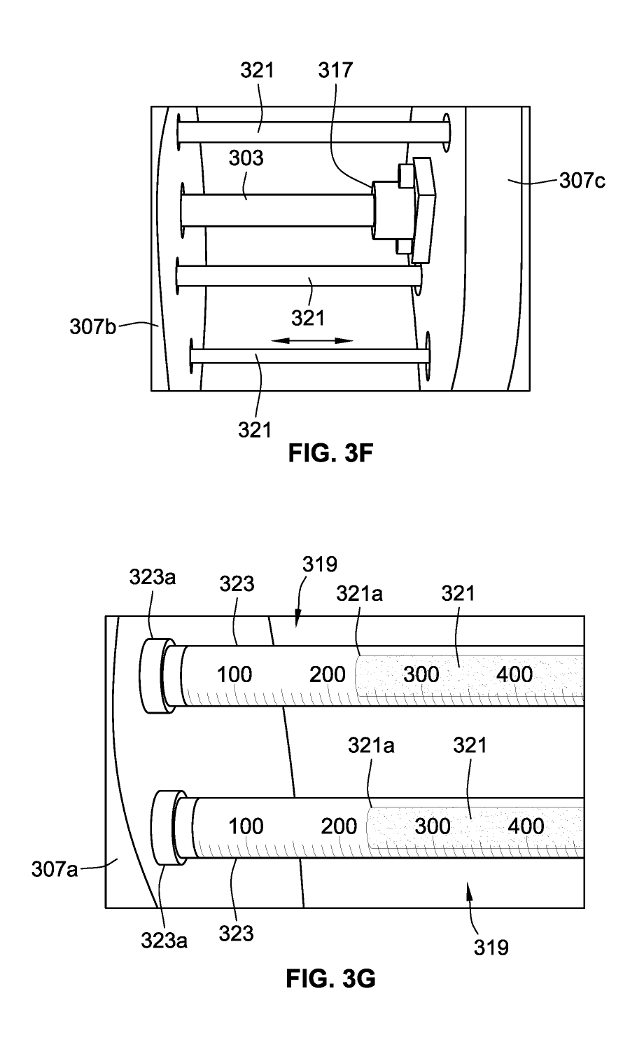

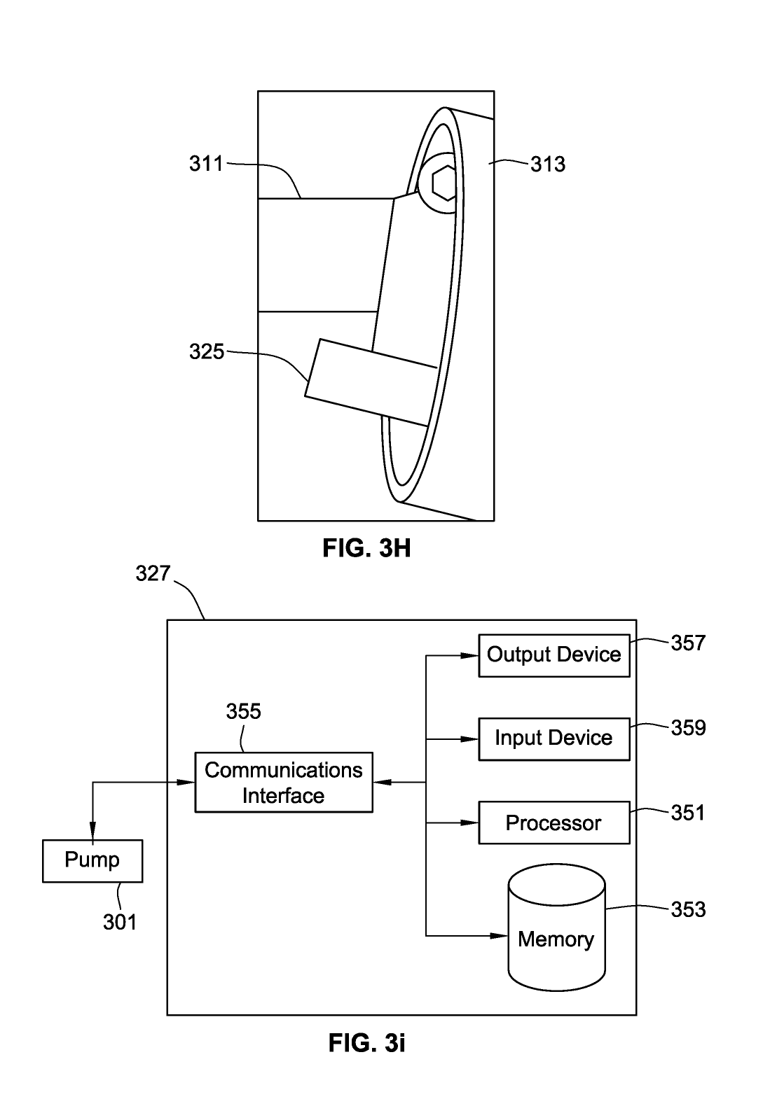

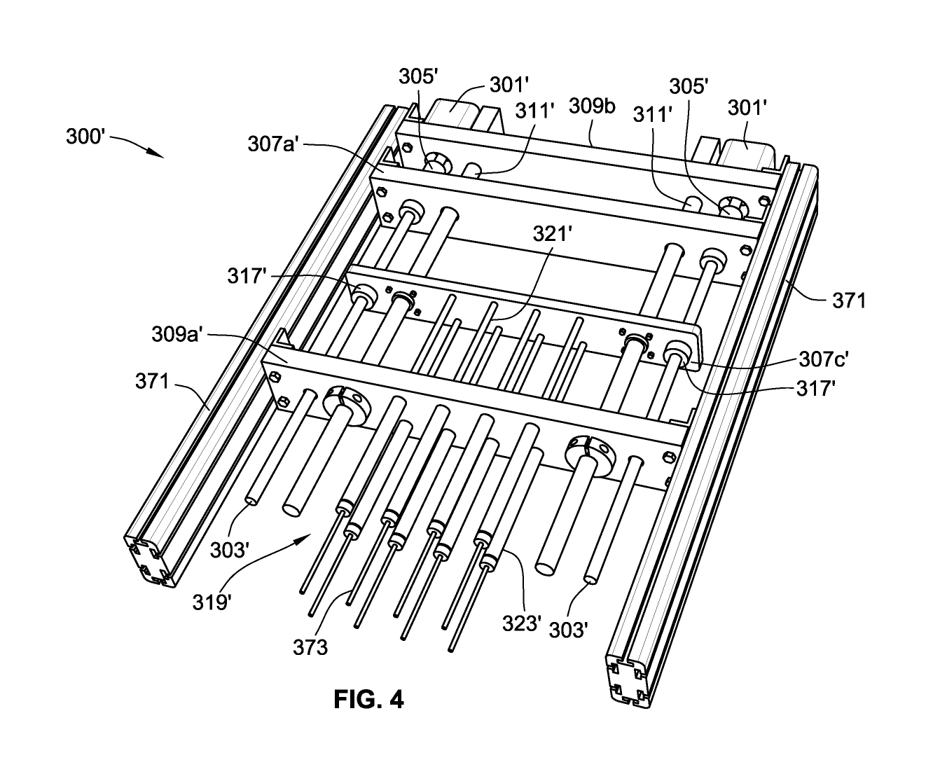

To provide the desired flow of fluid through at least one of the microchannel 34 and the microchannel 36 of the OOC 10, the present disclosure includes a fluid pump. Specifically, FIG. 3A is a perspective view of a fluid pump 300, in accord with some aspects of the present concepts. The fluid pump 300 is designed to cyclically pull a fluid (e.g., air) into the OOC 10 and subsequently push the fluid out of the OOC 10. With respect to the OOC 10 described above, and by way of an example, the fluid pump 300 can be connected to and in fluid communication with the OOC 10 through the fluid outlet 24. Operation of the fluid pump 300 creates suction through the fluid outlet 24 and the microchannel 34 that draws fluid into the OOC 10 through the fluid inlet 14. After entering the OOC 10, the fluid passes through the microchannel 34 and out of the OOC 10 through the fluid outlet 24. The fluid pump 300 then reverses the fluid flow to generate a bi-directional flow. Specifically, continued operation of the fluid pump 300 pushes fluid into the fluid outlet 24 and through the microchannel 34. After passing through the microchannel 34, the fluid passes out of the fluid inlet 14 to achieve the bi-directional flow of fluid through the microchannel 34. As understood based on the foregoing example, the fluid inlet 14 and the fluid outlet 24 can act as both fluid inlets and fluid outlets.

As shown FIG. 3A, the fluid pump 300 includes a motor 301. The motor 301 can be any type of motor that can power the fluid pump 300. According to some embodiments, the motor 301 is a stepper motor, and each step of the motor 301 can be controlled to deliver precise and accurate amounts of fluid to one or more OOCs 10.

As shown in FIG. 3A, and as shown in detail in FIG. 3B, the shaft 301a of the motor 301 is coupled to a lead screw 303 by a coupler 305. The lead screw 303 extends through plates 307a-307c and is coupled to an end plate 309a. Opposite the end plate 309a may be another end plate 309b at the opposite end of the fluid pump 300. As shown in detail in FIG. 3C, the motor 301 may be coupled to the end plate 309b. The plates 307a-307c and the end plates 309a and 309b form a rigid structure to support one or more elements of the fluid pump 300. The plates 307a-307c and the end plates 309a and 309b can be formed of any suitable material that provides the structural rigidity of the system, such as an acrylic.

The lead screw 303 can be coupled to the end plate 309a according to various mechanical structures. According to one embodiment, the lead screw 303 is coupled to the end plate 309a by a pair of clamp collars 331 and a pair of thrust bearings 333, as shown in detail in FIG. 3D. The clamp collars 331 and thrust bearings 333 constrain the lead screw 303 to a single, rotational degree of freedom at the end plate 309a.

On both sides of the lead screw 303 are guide rails 311. The guide rails 311 pass through the plates 307a-307c and are fixed to the end plates 309a and 309b to add to the structural rigidity of the fluid pump 300. The plates 307a and 307b may be fixed within the fluid pump 300. Accordingly, the guide rails 311 can be fixed to the plates 307a and 307b by pairs of rubber clamps 313 supported by the guide rails 311 and on both sides of the plates 307a and 307b, where the plates 307a and 307b meet the guide rails 311. The rubber clamps 313 can be formed of, for example, rubber gaskets and shaft collars to provide additional rigidity within the fluid pump 300.

The guide rails 311 also pass through the plate 307c. According to the illustration shown in FIG. 3A, the plate 307c is movable about the guide rails 311. Accordingly, the plate 307c couples to the guide rails 311 by way of linear bearings 315. As shown in a detailed view in FIG. 3E, the guide rail 311 passes through the center of the linear bearing 315. The linear bearing 315 can be any type of linear bearing, such as a linear ball bearing, a linear bushing bearing, etc. The linear bearings 315 provide proper alignment of the various components within the fluid pump 300.

The plate 307c is coupled to the lead screw 303 by a traveling nut 317, as shown in detail in FIG. 3F. The traveling nut 317 is rotationally fixed with respect to the plate 307c. The plate 307c is rotationally fixed within the fluid pump 300 by being coupled to the guide rails 311 through the linear bearings 315. Accordingly, rotation of the lead screw 303 by the motor 301 causes the traveling nut 317 to translate about the lead screw 303 in the directions represented by the arrow in FIG. 3F.

The fluid pump 300 further contains syringes 319, as shown at least partially in detail in FIG. 3G. By way of example, and without limitation, the fluid pump 300 can include four syringes 319; however, the number of syringes 319 can vary without departing from the spirit and scope of the present disclosure. The syringes 319 can be various sizes depending on the overall desired size of the fluid pump 300, the amount of fluid to be pumped, etc. In some aspects, the syringes 319 can be 500 μl glass syringes. The syringes 319 are gas tight and can deliver fluid according to precise and accurate measurements based on the displaced volume within the syringes 319. That is, the syringes 319 include moveable plungers 321 and fixed barrels 323, with the movable plungers 321 being supported within the barrels 323. Ends 323a of the barrels 323 attach to the plate 307a. The ends 323a can be permanently fixed or removably coupled to the plate 307a. By way of example, the plate 307a can include gaskets, such as O-rings (not shown), that couple to the ends 323a of the barrels 323 and allow the ends 323a to be selectively decoupled from the plate 307a.

Opposing ends 323b of the barrels 323 attach to the plate 307b. The ends 323b can be permanently fixed or removably coupled to the plate 307b. By way of example, the plate 307b can include gaskets (not shown) built within the plate 307b that couple to the ends 323b of the barrels 323 and allow the ends 323b to be selectively decoupled from the plate 307b.

Ends 321a of the plungers 321 are inserted into the barrels 323. Opposing ends 321b of the plungers 321 are coupled to the plate 307c. The ends 321b can be permanently fixed or removably coupled to the plate 307c. By way of example, the plate 307c can include gaskets (not shown) built within the plate 307c that couple to the ends 321b of the plungers 321 and allow the ends 321b to be selectively decoupled from the plate 307c.

According to the ends 321b of the plungers 321 being coupled to the plate 307c, and the plate 307c being coupled to the traveling nut 317, operation of the motor 301 rotates the lead screw 303, which causes the traveling nut 317, the plate 307c, and the plungers 321 to translate relative to the barrels 323 (which are fixed within the fluid pump 300). Thus, operation of the motor 301 actuates the syringes 319. Depending on the direction of the actuation, the plungers 321 cause fluid to be drawn in or pushed out of the syringes 319. With the syringes 319 in fluid communication with other elements, such as one or more OOC 10, the syringes 319 draw in and push out fluid in the microchannels of one or more of the OOCs 10. Thus, the fluid pump 300, with the motor 301 actuating the syringes 319, allows for a precise and accurate control of bi-directional fluid flow through one or more OOCs 10.

The amount of fluid drawn in and pushed out of the OOC 10 can depend on several factors. One factor that controls the amount of moved fluid is the size of the syringes 319, both with respect to length and diameter. By way of example, and without limitation, the syringes 319 can usually have a length of 1 to 20 cm, such as 10 cm, and can have a diameter of 1 to 50 mm, such as 3.26 mm.

A second factor that controls the amount of moved fluid is the amount of actuation of the syringes 319 by the motor 301. By way of example, and without limitation, the motor 301 can cause the plungers 321 to actuate 1 to 20 cm within the barrels 323, such as 3 cm. Based on at least the above two factors, the fluid pump 300 is designed to provide precise and accurate control of an amount of fluid to the OOC 10.

The fluid pump 300 further includes a limit switch 325, as shown in detail in FIG. 3H. The limit switch 325 indicates an end of one cycle of operation of the fluid pump 300. By way of example, and without limitation, the limit switch 325 can be configured to be triggered upon the plate 307c contacting the limit switch 325 to indicate the end of the traveling nut 317 and the plate 307c translating back and forth during a single period.

The motor 301 of the fluid pump 300 is connected to a controller 327 by a cable 329 (also shown in detail in FIG. 3C). FIG. 3I shows an illustrative controller 327 that can be employed to control the motor 301 of the pump, in accord with some aspects of the present concepts. The controller 327 comprises one or more processors 351 communicatively coupled to memory 353, one or more communications interfaces 355, and one or more output devices 357 (e.g., one or more display devices), and one or more input devices 357.

The memory 353 can include any computer-readable storage media, and can store processor-executable instructions, such as computer instructions, for implementing the various functionalities described herein for the respective systems, as well as any data relating thereto, generated thereby, or received via the communications interface(s) or input device(s).

The processor(s) 351 shown in FIG. 3H can be used to execute the instructions stored in the memory 353 and, in so doing, also can read from and/or write to the memory 353 various information processed and/or generated pursuant to execution of the instructions.

The processor(s) 351 of the controller 327 also may be communicatively coupled to or control the communications interface 355 to transmit or receive various information pursuant to execution of instructions. For example, the communications interface 355 may be coupled to a wired or wireless network, bus, or other communication medium, such as the cable 329, and may, therefore, allow the controller 327 to transmit information to and/or receive information to the motor 301. In some implementations, the communications interfaces 355 may be configured (e.g., via various hardware components or software components) to provide a website or applications program (an application) on a handheld device as an access portal to at least some aspects of the controller 327. Non-limiting examples of such hand-held devices are tablets, slates, smartphones, electronic readers, or other similar hand-held electronic devices.

The output devices 357 of the controller 327 present various information in connection with execution of the instructions. The input devices 357 permit a user to make manual adjustments, selections, enter data, and interact in any of a variety of manners with the processor 351 during execution of the instructions.

By way of example, and without limitation, the controller 327 allows an operator of the fluid pump 300 to program various rhythms and volumes for the flow of fluid into and out of the OOC 10. The rhythm can be a continuous or intermittent oscillation of fluid into and out of the OOC 10. The volume of fluid for each cycle of drawing fluid into and out of the OOC 10 can be the same volume of fluid or a different volume of fluid. Various profiles of the controller 327 can operate the fluid pump 300 to mimic various breathing patterns in humans. By way of example, and without limitation, one profile can mimic the breathing of a human during rest. The controller 327 controls the motor 301 such that the period of the actuating the syringes 319 and the volume of fluid moved by the syringes 319 mimics the period and volume of a human during rest. Another profile can be breathing during exercise or exertion, such as a shorter period and larger volumes of fluid. Thus, the controller 327 causes the fluid pump 300 to generate different shear stress levels on the cells lining the microchannels 34 and/or 36 within the OOC 10, with the fluid pump 300 connected to the OOC 10 through one or more of the microchannels 34 and/or 36.

In summary, the controller 327 controls the duration of each aspect of the cycle (intake duration and output duration), and the volume of air that is moved within each the cycle. More specifically, the controller 327 can control the intake and outtake duration and volume, the duration between puffs, the number of puffs per cigarette, and the number of cigarettes smoked, etc. Furthermore, as discussed below, the controller 327 may be used to control the temperature of the fluids used in the cycles, as well as the content of the fluid (e.g., introduction of environmental contaminants, such as smoke, or therapeutic agents, such as medication) in the cycles.

Based on the various profiles, the fluid pump 300 allows for the exposure of airway epithelial cells within the OOC 10 to rhythmic air flow to mimic breathing where mechanical cues of air transfer (e.g., shear stress) can be recapitulated. The fluid pump 300 also allows for the reconstruction of tissue/organ damage due to fluid-mechanical movements, such as airway damage due to the collapse and opening of airway tubes, according to the cyclic passage of fluid over the cells in the OOC 10 to mimic ventilation-induced lung injury.

Based on combining the fluid pump 300 with one or more of the OOCs 10, the fluid pump 300 can flow fluid into and out of each OOC 10. According to some embodiments, each syringe 319 of the fluid pump 300 can connect to a separate OOC 10. Alternatively, multiple syringes 319 can connect to a single OOC 10, depending on the volume of fluid that is desired through the OOC 10. By way of example, and without limitation, a syringe 319 of the fluid pump 300 can connect to the fluid outlet 24 of the OOC 10. A fluid supply, such as air, can be connected to the fluid inlet 14. Accordingly, actuation of the syringe 319 causes air to flow through the microchannel 34 in a first direction, and then the air to flow back through the microchannel 34 in a second, opposite direction to the first direction. The flow of air in the first and second direction mimics the flow of air through the respiratory system of a human during respiration. The volume of air through the microchannel channel 34 can be accurately and precisely controlled based on controlling the motor 301 with the fluid pump 300.

The above described system of the fluid pump 300 connected to one or more OOC 10 is one embodiment of a system for testing the effects of fluid flow on cells lining the microchannels 34 and/or 36 of the OOC 10. One or more elements can be added to the system to further mimic the effects of fluid flow, in addition to mimicking other effects on the OOC 10, such as introducing one or more agents into the fluid for mimicking the effects of the one or more agents on the OOC 10.

FIG. 4 is a perspective view of an alternative configuration of the fluid pump 300 of FIG. 3A, in accord with some aspects of the present concepts. Namely, FIG. 4 shows a fluid pump 300′, with similar reference numerals referring to similar features discussed above. Specifically, the fluid pump 300′ includes two motors 301′. Like the motor 301 discussed above, the motors 301′ can be any type of motors that can power the fluid pump 300′, such as stepper motors. As stepper motors, each step of the motors 301′ can be controlled to deliver precise and accurate amounts of fluid to one or more OOCs 10 fluidly connected to the fluid pump 300′.

Each motor 301′ is connected to end plates 309a′ and 309b′ by guide rails 311′. Further, the fluid pump 300′ includes side rails 371 that are connected at least to the guide rails 311′. The side rails 371 can also be directly connected to the motors 301′ and/or the plate 307a′. The side rails 371 allow for the fluid pump 300′ to be inserted within and coupled to, for example, an incubator chamber, such as chamber 509a discussed below.

Each motor 301′ is connected to a separate lead screw 303′ through a separate coupler 305′. The couplers 305′ extend through the end plate 309b′, and the lead screws 303′ extend through the end plate 309a′, in addition to plates 307a′ and 307c′. Similar to the fluid pump 300, the plate 307a′ can be fixed or stationary within the fluid pump 300′. The plate 307a′ can be connected to the sides rails 371 and/or fixed to the guide rails 311′.

The plate 307c′ is configured to move within the fluid pump 300′. Specifically, each lead screw 303′ connects to the plate 307c′ through a separate traveling nut 317′. Accordingly, as discussed above, activation of the motors 301′ cause the couplers 305′ and lead screws 303′ to rotate. The traveling nuts 317′ convert the rotational movement into linear movement of the plate 307c′ along the guide rails 311′.

The plate 307c′ is connected to one or more syringes 319′. The linear movement of the plate 307c′ causes the syringes 319′ to either take in or pump out fluid, as discussed above. In greater detail, the plate 307c′ is connected to plungers 321′ of the syringes 319′. Each plunger 321′ is inserted into a separate barrel 323 for each syringe 319′. The barrels 323′ are fixed to the end plate 309a′ to remain stationary as the plungers 321′ are actuated within the barrels 323′. Opposite ends of the barrels 323′ from the plungers 321′ are connected to fluid lines 373 (e.g., tubing 511f discussed below) that supply fluid to the one or more OOC 10.

The operation of the fluid pump 300′ is generally the same as the fluid pump 300. The fluid pump 300′ can, therefore, vary according to the variations discussed above with respect to the fluid pump 300, such as vary whether the plungers 321′ or the barrels 323′ are movable relative to the fixed components of the fluid pump 300′. Further, although eight syringes 319′ are shown in the fluid pump 300,′ there can be less or more syringes depending on, for example, the amount of fluid desired to be pumped and/or the number of OOC 10 that are desired to be connected to the fluid pump 300′. The general differences between the fluid pump 300 and the fluid pump 300′ include that the fluid pump 300′ presents a slimmer profile than the fluid pump 300. The slimmer profile allows the fluid pump 300′ to be contained in smaller areas, such as smaller chambers, or take up less space within the same size chambers. Further, another general difference is that the fluid pump 300′ includes the side rails 371. The side rails 371 can be configured to mate with corresponding rails within chambers for easy insertion and connection of the fluid pump 300′ within and to a chamber.

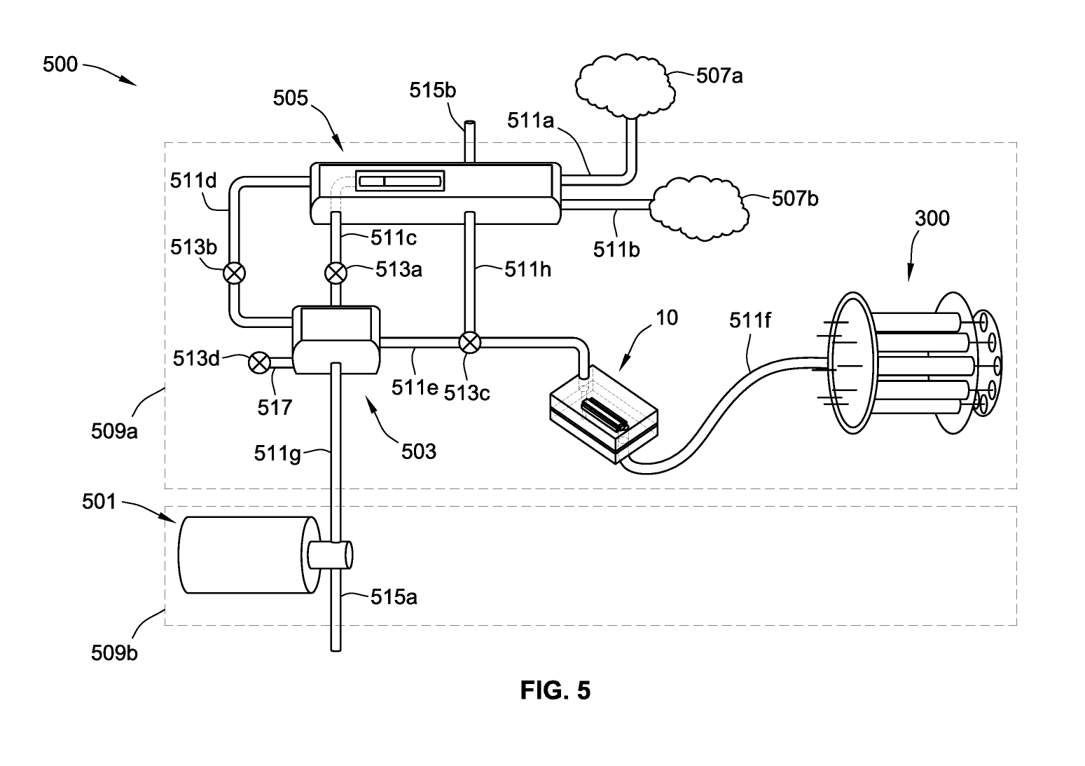

FIG. 5 shows a system 500 for introducing fluid and, more particularly, fluid mixed with one or more agents, through the OOC 10, in accord with aspects of the present concepts. As will be described in greater detail below, the system 500 includes the OOC 10 in fluid communication with the fluid pump 300. Although only a single OOC 10 and a single fluid pump 300 are shown in FIG. 5, the system 500 can include multiple OOCs 10 and/or multiple fluid pumps 300. Additionally, according to some embodiments, the system 500 can include one or more peristaltic pumps, compressor pumps, diaphragm pumps, and/or piston pumps in addition, or in the alternative, to the fluid pump 300 for moving fluid through the OOC 10.

In general, the system 500 includes a suction pump 501, a reservoir 503, and a mixer 505. According to some embodiments, the mixer 505 is connected to a fluid source 507a outside of a chamber 509a housing the mixer 505, the reservoir 503, the OOC 10, and the suction pump 501. Alternatively, or in addition, the mixer 505 is connected to a fluid source 507b within the chamber 509a. The mixer 505 is connected to the fluid source 507a by tubing 511a and to the fluid source 507b by tubing 511b. Alternatively, the mixer 505 can be connected to the fluid source 507b through an inlet port (not shown) built into the mixer 505 without including the tubing 511b. As will be described in greater detail below, the mixer 505 introduces one or more agents into the fluid from the fluid source 507a and/or the fluid source 507b for analyzing the effects of the one or more agents on the OOC 10 within the system 500.

The reservoir 503 is connected to the mixer 505 by tubing 511c. Alternatively, or in addition, the reservoir 503 is connected to the mixer 505 by tubing 511d. However, according to some embodiments, the tubing 511d can instead attach to an exhaust line 515b. The system 500 can include valves 513a and 513b, such as two-way pinch valves, along the tubing 511c and 511d, respectively, to control the flow of fluid, or fluid mixed with one or more agents, from the mixer 505 to the reservoir 503. The reservoir 503 is also connected to the OOC 10 by tubing 511e, and the OOC 10 is connected to the fluid pump 300 by tubing 511f. The reservoir 503 is also connected to the suction pump 501 by tubing 511g.

According to some embodiments, and as illustrated in FIG. 5, the suction pump 501 is located within the chamber 509b, which is separate from the chamber 509a. However, according to some embodiments, the suction pump 501 can be located in the chamber 509a, and the chamber 509b can be omitted from the system 500. The suction pump 501 includes an exhaust line 515a, which allows the fluid, or fluid mixed with one or more agents, to exit the chamber 509b, or chamber 509a, if the chamber 509b is omitted from the system 500. According to some embodiments, the mixer 505 can include an exhaust line 515b, which allows fluid and, potentially, fluid mixed with one or more agents, to exit the mixer 505 and the chamber 509a.

According to the above arrangement, the suction pump 501 generates a vacuum or suction within the system 500 to move fluid, at least in part, through the system 500. Initially, the suction generated by the suction pump 501 draws fluid into the mixer 505 from the fluid source 507a, the fluid source 507b, or both. The fluid sources 507a and 507b can be any type of fluid source that supply a fluid to the system 500. The fluid can be a gas and/or a liquid. According to some embodiments, the fluid can be a gas or a liquid that includes particulates (e.g., nano-particulates) suspended within the fluid and that can flow through the system 500. By way of example, and without limitation, the fluid of the fluid source 507a and the fluid source 507b can be air. The fluid source 507a, therefore, can be air from the environment outside of the chamber 509a, and the fluid source 507b, therefore, can be air from the environment inside of the chamber 509b. Although the fluid within the system 500 is primarily described throughout as being air, the fluid can constitute any fluid for which experiments on the OOC 10 are desired.

The fluid then flows into the mixer 505, which can also be referred to herein as an agent introduction apparatus. Within the system 500, the mixer 505 introduces one or more agents into the fluid. By way of example, and without limitation, upon air entering the mixer 505, the air is mixed with one or more agents. The one or more agents can be any additional fluid (e.g., liquid or gas), or particulates (e.g., nano-particulates) alone or suspended within another fluid, for which experiments on the OOC 10 are desired. With lung cells lining the microchannels 34 and 36 of the OOC 10, the one or more agents introduced into the fluid by the mixer 505 can be any agent for determining the effects of the agent on the lung cells. By way of example, and without limitation, the one or more agents can include smoke, such as from burning tobacco products, construction materials, wood, cooking grease, fuel (e.g., gasoline, diesel, etc.)—which can include both the gaseous phase and the solid phase (e.g., nano-particulates) of the smoke—aerosols, liquid and/or gaseous chemicals (e.g., carbon monoxide, carbon dioxide, nitrogen, chlorine, hydrogen cyanide, etc.), medication (e.g., systemic corticosteroids, anticholinergics, short-acting beta-agonists, antihistamines, etc.), volatile organic compounds (VOCs), pollen, mold and/or plant spores, bacteria, viruses, dust, dust mites, smog, animal dander, lead paint dust, etc.

The configuration of the mixer 505 can vary depending on the one or more agents to be mixed with the fluid. For one or more agents that do not require a chemical reaction or transformation (e.g., burning) to occur prior to the mixing, the mixer 505 can be, for example, a chamber that includes an apparatus for releasing the one or more agents into the fluid at a controlled amount and/or rate. Such as apparatus can include a pressurized source of the one or more agents, such as a pressurized can or cylinder. The one or more agents are then released into the incoming fluid in a controlled manner to obtain a desired concentration of the one or more agents within the fluid, such as a controlled release of medication, VOCs, and/or carbon dioxide.

For one or more agents that are associated with and/or require a chemical reaction or transformation occurring prior to the mixing, the mixer 505 can be a specialized apparatus for controlling the chemical reaction and/or transformation, such as a smoking apparatus. Such a chemical transformation can include burning one or more sources to generate the one or more agents. By way of example, and without limitation, the one or more sources can include fuel (e.g., gasoline, diesel, etc.) or tobacco products, and the specialized apparatus can control the burning of the fuel or tobacco products to generate the one or more agents. In the case of burning, the one or more agents include the gaseous and nano-particulate phases of the resulting smoke.

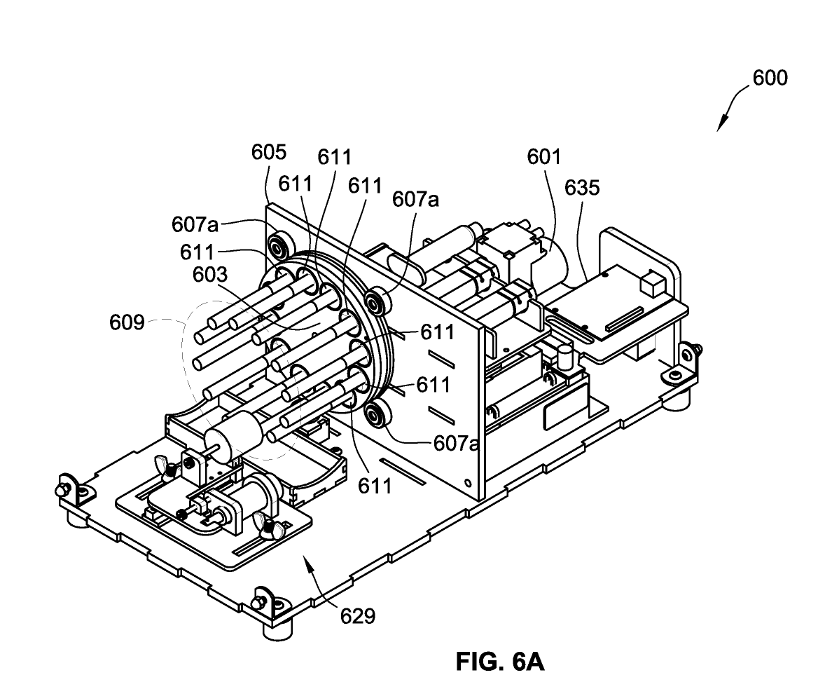

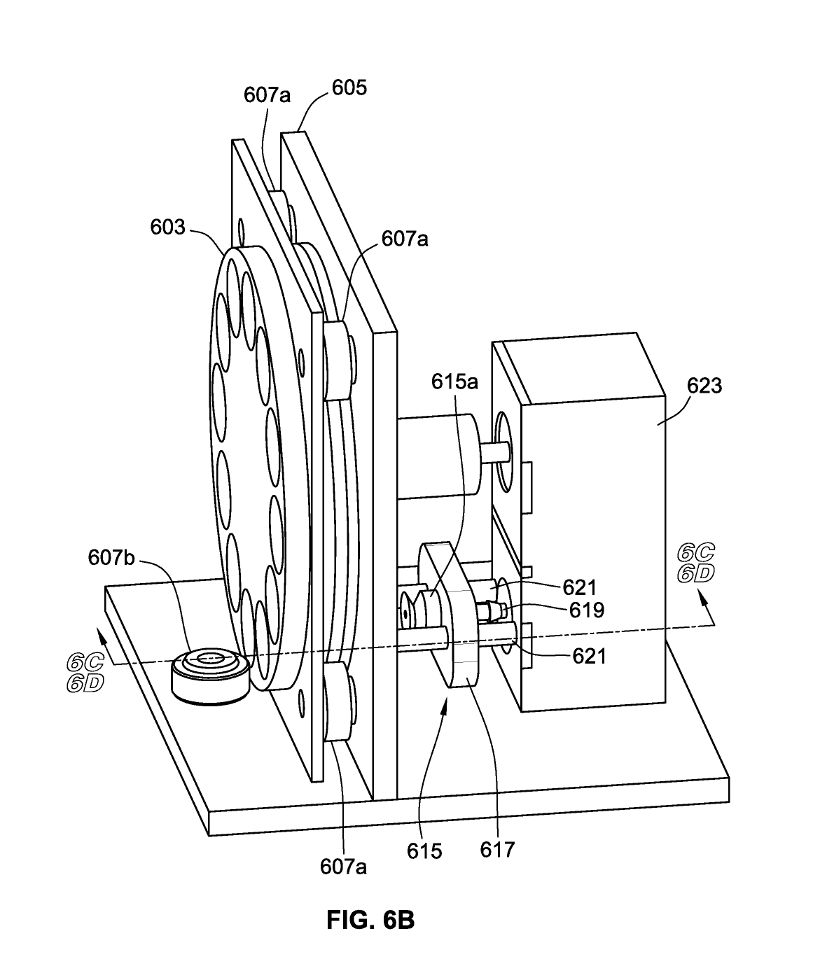

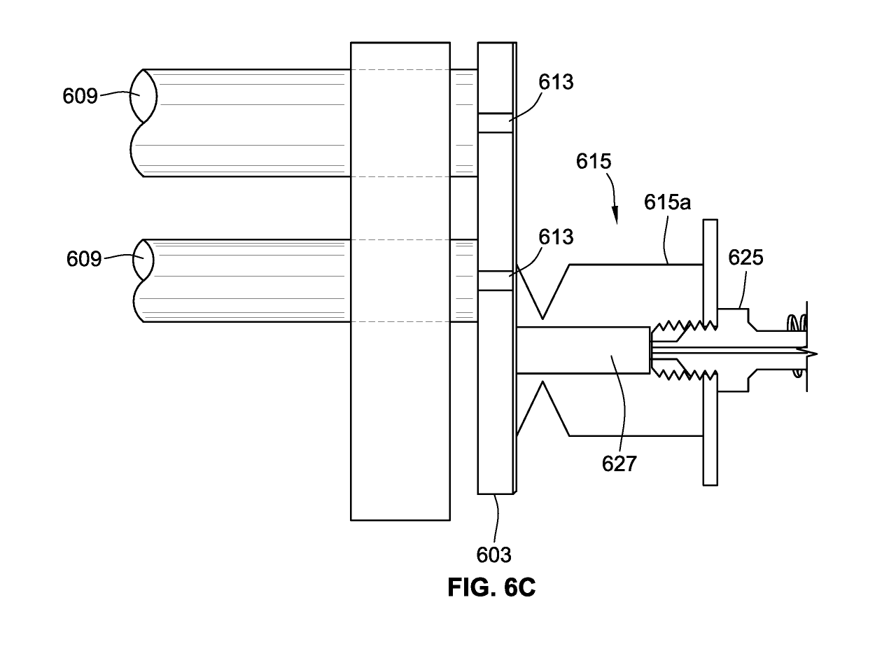

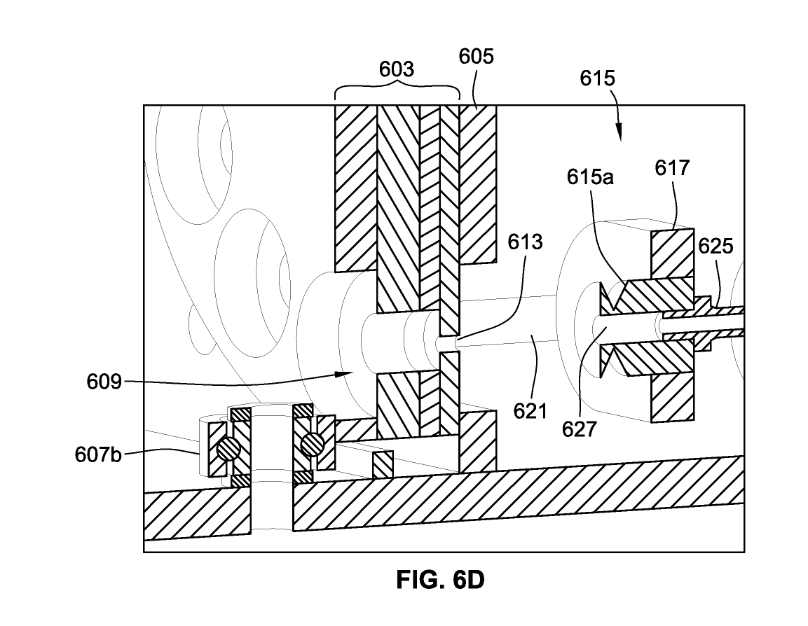

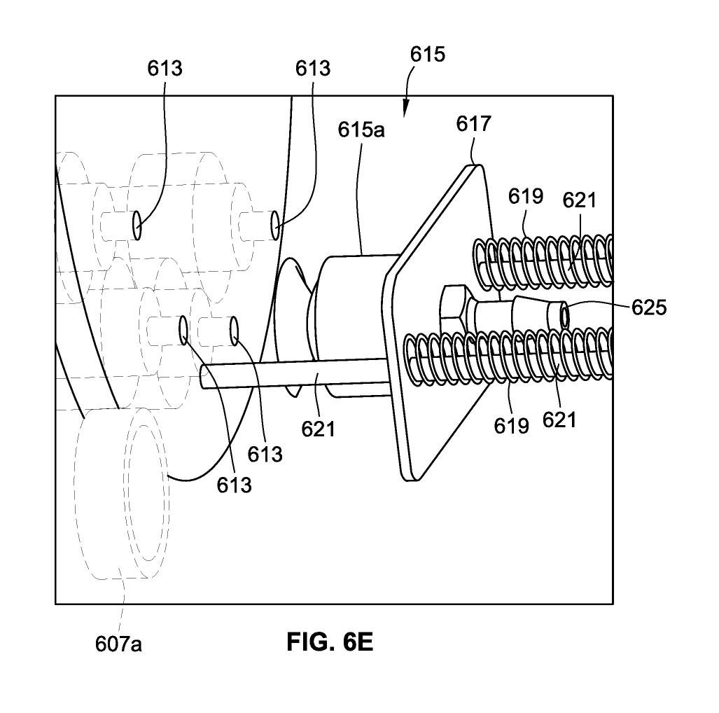

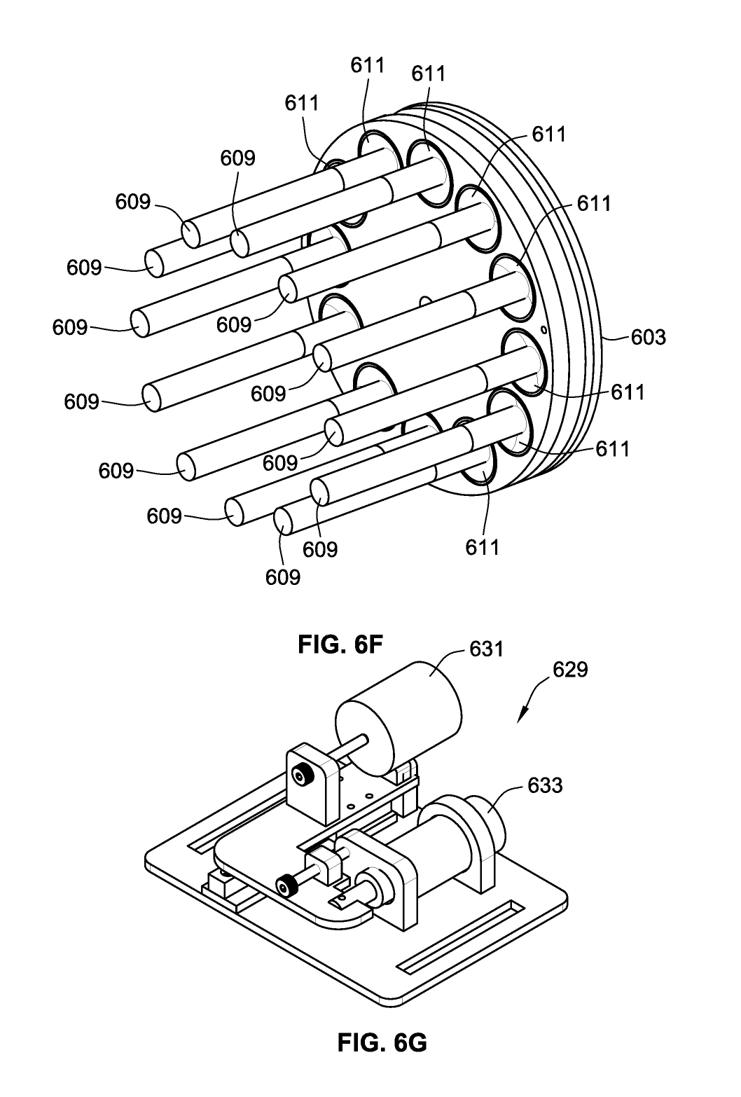

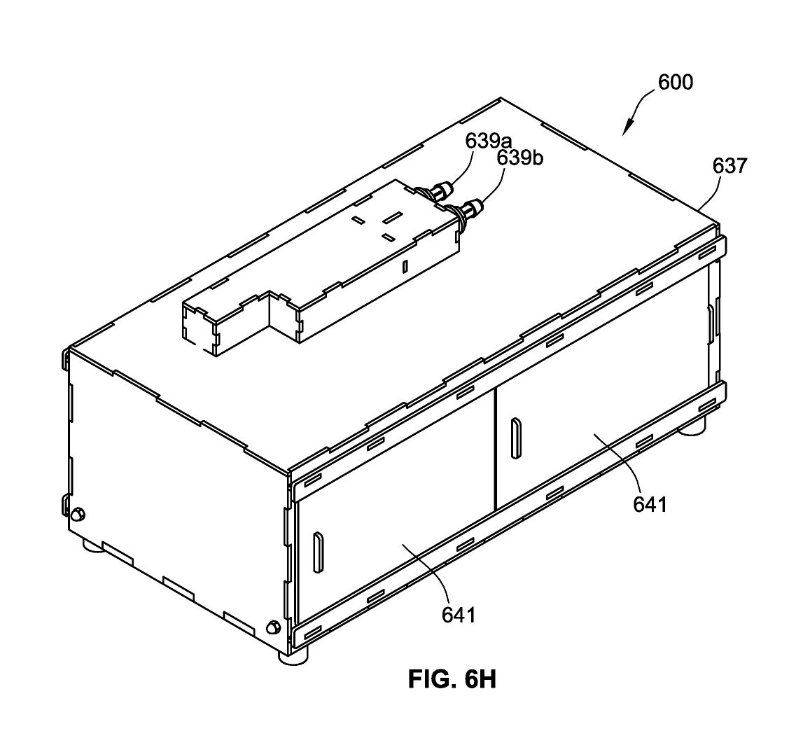

According to some embodiments, and as described in greater detail below with respect to FIG. 6A, the mixer 505 can be a smoking apparatus that generates smoke, such as by a controlled burning one or more smokeable products. With a smoking apparatus as the mixer 505 within the system 500, the smoking apparatus provides for a controlled smoking of one or more smokeable products. The mixer 505, configured as the smoking apparatus, can include the tubing 511c configured to connect directly to the smokeable product for generating first-hand smoke. The mixer 505 also can include the tubing 511d configured to connect to the mixer 505 for drawing out second-hand smoke from within the mixer 505. With the tubing 511c and the 511d, both first-hand smoke and second-hand smoke can be drawn from the mixer 505 for passing through the OOC 10. However, for a system 500 designed specifically for analyzing one of first-hand smoke or second-hand smoke, tubing 511d or tubing 511c, respectively, can be removed.

A distinction is made between “first-hand” and “second-hand” smoke. First-hand smoke refers to the fluid mixed with smoke that passes out of the smokeable product in a mixed state. Second-hand smoke refers to the smoke that comes off of the smokeable product and subsequently mixes with the fluid, such as within the mixer 505. Accordingly, first-hand smoke is used in a similar context as first-hand smoke in referring to smoke that is drawn directly into the lungs through, for example, a user smoking a cigarette, rather than from the atmosphere surrounding a cigarette.

According to some embodiments, the system 500 can also include tubing 511h connecting the mixer 505 directly to the tubing 511e (i.e., bypassing the reservoir 503). The tubing 511h can connect to the tubing 511e at a valve 513c. Like the tubing 511d, the tubing 511h can draw second-hand smoke from the mixer 505. However, the tubing 511h bypassing the reservoir 503 can bypass the second-hand smoke from mixing with any non-second-hand smoke (e.g., first-hand smoke or fresh air) that may be in the reservoir 503.

From the mixer 505, the fluid mixed with the one or more agents flows into the reservoir 503. The reservoir 503 is an airtight chamber that is formed of, for example, an acrylic. Here, the fluid mixed with the one or more agents can collect and further mix prior to either being drawn into the OOC 10 based on the operation of the fluid pump 300 or being drawn out of the system 500 at exhaust line 515a after passing through the suction pump 501. The dimensions of the reservoir 503 can be configured based on the reservoir 503 mimicking the volume within an average person's lungs or pulmonary system. Further, the dimensions of the reservoir 503 can be configured such that a desired volume of fluid mixed with one or more agents flows into the OOC 10 based on the operation of the fluid pump 300 drawing a percentage or amount of the fluid from the reservoir 503 into the OOC 10. According to some embodiments, the reservoir 503 has a volume of 1 to 1000 ml, such as 100 ml.

According to some embodiments, the reservoir 503 can include an inlet port 517. The inlet port 517 can be selectively opened and closed by actuation of a valve 513d, such as a two-way pinch valve, to allow for fluid to enter the reservoir 503 from an alternative fluid source than the fluid source 507a and the fluid source 507b. Alternatively, the inlet port 517 allows fluid from the fluid source 507b (e.g., air within the chamber 509a) to enter the reservoir 503, or from the fluid source 507a (e.g., air outside of the chamber 509a) to enter the reservoir 503. According to the reservoir 503 including the inlet port 517, fluid mixed with the one or more agents within the reservoir 503 can be drawn out through the suction pump 501 and replaced with fluid not mixed with the one or agents by bypassing the mixer 505.

Although shown as including only one reservoir 503, according to some embodiments, the system 500 can include multiple reservoirs 503. By way of example, and without limitation, one reservoir 503 can function as a first-hand smoke reservoir, a second reservoir 503 can function as a second-hand smoke reservoir, and a third reservoir 503 can function as a reservoir 503 without the fluid mixed with the one or more agents (e.g., a fresh air reservoir).

After the reservoir 503, and before being expelled out of the system 500, the fluid mixed with the one or more agents passes through the suction pump 501. Alternatively, according to some embodiments, the fluid mixed with the one or more agents can be transported to outside of the system 500, such as without passing through the suction pump 501. By way of example, upon actuation of the fluid pump 300 and the opening of an exhaust line immediately after the OOC 10 (not shown), the fluid mixed with the one or more agents can be expelled out of the system 500 under the force of the fluid pump 300. Alternatively, the fluid mixed with the one or more agents can be transported to other elements within the system 500. As discussed above, the system 500 can include multiple OOCs 10 (e.g., a first OOC 10 and a second OOC 10). By way of example, and without limitation, the fluid mixed with the one or more agents can be transported to the second OOC 10 after exiting the first OOC 10. For example, the fluid mixed with the one or more agents can be transferred to the second OOC 10 within an enclosure to be bubbled through a cell culture medium to generate, for example, cigarette smoke extract for stimulation of endothelial cells, myocytes, etc. Alternatively, the second OOC 10 can have different cells lining, for example, the microchannel 34. For example, the first OOC 10 can have cells lining the microchannel 34 that correspond to cells of the trachea, and the second OOC 10 can have cells lining the microchannel 34 that correspond to cells of the bronchus. Additional OOCs 10 can be added in series that, for example, eventually include cells lining the microchannel 34 that correspond to pulmonary alveolus cells. Accordingly, multiple OOCs 10 can be added in series, with each OOC 10 corresponding to different cell types, to represent the entire pulmonary system within the system 500.

Referring back to the fluid mixed with the one or more agents exiting the system 500 after passing through the suction pump 501, to prevent clogging caused by the one or more agents mixed with the fluid (e.g., such as by the nano-particulates), the suction pump 501 can be a valve-less diaphragm ceramic pump. However, the suction pump 501 can be other types of pumps capable of providing a vacuum or suction within the system 500, including pumps with valves, without departing from the spirit and scope of the present disclosure. Further, by placing the suction pump 501 at the end of the flow of fluid mixed with the one or more agents within the system 500, nano-particulates included in the one or more agents that may otherwise cause the suction pump 501 to become clogged are trapped elsewhere, such as in the reservoir 503, before reaching the suction pump 501. Additionally, by including the suction pump 501 at the end of the flow within the system 500, sources of one or more agents, such as multiple cigarettes, can be introduced (e.g., smoked) either simultaneously or in series, with the suction being provided by the suction pump 501 for all of the various sources. By way of example, and without limitation, according to the setup of FIG. 5, multiple cigarettes (as will be discussed in greater detail below in FIG. 6A) can be smoked at the same time. Accordingly, whole cigarette smoke can be introduced into the OOC 10 as opposed to, for example, cigarette smoke extract (CSE) or cigarette smoke condensate (CSC).

According to some embodiments, the system 500 can include a filter and/or a scrubber (not shown) between the reservoir 503 and the suction pump 501 to remove one or more agents that would clog the suction pump 501, or one or more agents that are toxic and must be removed from the exhaust prior to exiting the system 500.

As discussed above, the mixer 505, the reservoir 503, the OOC 10, and the fluid pump 300 can be within the chamber 509a. According to some embodiments, the chamber 509a can be an incubator that allows for control of the conditions inside of the chamber 509a. By way of example, and without limitation, the chamber 509a can be operated at a set temperature, such as 37° C., which mimics the internal temperature of the human body. The chamber 509a can also be operated at other conditions that mimic the internal conditions of a human body. By way of example, and without limitation, such other conditions include humidity that mimics the humidity within the lungs and/or pulmonary system of the human body, such as 95-100% humidity. By being inside of the chamber 509a operated at conditions that mimic the internal conditions of a human, such as within the lungs and the pulmonary system, the mixer 505, the reservoir 503, the OOC 10, and the fluid pump 300 are operated at conditions that mimic the conditions in vivo.

With respect to the fluid source 507b discussed above, because the fluid source 507b is within the chamber 509a, the fluid of the fluid source 507b is also at the conditions within the chamber 509a. By way of example, and without limitation, air as the fluid within the system 500 is at the conditions within the chamber 509a, such as 37° C., when the air enters the mixer 505. Additionally, with the fluid source 507a outside of the chamber 509a, the fluid within the fluid source 507a can be at different conditions than the conditions within the chamber 509b. For example, the conditions outside of the chamber 509a can be different environmental conditions that a user might experience in the real world, such as a temperature between −40 to 50° C., such as 0° C., and/or a humidity between 0 to 100% humidity, such as 50% humidity. Based on the different conditions between inside and outside of the chamber 509a, and the ability to select a fluid source at different or the same conditions as inside the chamber 509a, the effect of transitions in conditions of a fluid source to the conditions inside the chamber 509a can be analyzed within the system 500. By way of example, and without limitation, the effect of a smoker smoking outside on a cold day versus a hot day can be analyzed based on the different conditions of the fluid source 507a outside of the chamber 509a.

As discussed above, according to some embodiments, the suction pump 501 is located outside of the chamber 509a, such as within the separate chamber 509b. According to some embodiments, the chamber 509b also can be an incubator that allows for control of the conditions inside of the chamber 509b. By way of example, and without limitation, the chamber 509b can also be operated at set environmental conditions, such as at a set temperature and/or a set humidity. By way of example, and without limitation, the temperature within the chamber 509b can be controlled to mimic conditions outside of the human body, such as 25° C. and ambient humidity conditions. However, according to some embodiments, the suction pump 501 can be located within the chamber 509a. Alternatively, the suction pump 501 can be located within the chamber 509b, and the chamber 509b can be set to the same operating conditions as the chamber 509a.

According to the arrangement shown in FIG. 5, one or more agents can be introduced into a fluid. The fluid mixed with the one or more agents can then be passed through the OOC 10 based on the operation of the fluid pump 300. The OOC 10 can then be analyzed for the effects of the one or more agents on, for example, the lung cells lining the microchannels 34 or 36 of the OOC 10. The system 500 provides an arrangement that mimics the in vivo conditions of the lungs and/or pulmonary system of a human, including the cyclical shear stress experienced in the passage ways within the lungs and the pulmonary system. Accordingly, the system 500 can accurately reproduce in vivo conditions to accurately reflect the effects of the one or more agents on the OOC 10.

As discussed above, the mixer 505 can be various specialized equipment depending on the specific one or more agents that are to be introduced into the fluid within the system 500. To examine the effects of smoke, particularly smoke from tobacco or related products (e.g., smokeable products), the mixer 505 can be a smoking apparatus that allows for one or more smokeable products to be burnt (e.g., smoked) for introducing one or more agents from the smokeable products into the fluid. Various smoking apparatuses currently exist that can be used as the mixer 505. However, FIGS. 6A-6H show various view and elements of a specific smoking apparatus 600 that can be used within the system 500 as the mixer 505, which allows for the controlled burning of multiple smokeable products, concurrently or consecutively.

Referring to FIG. 6A, the smoking apparatus includes a motor 601. The motor 601 can be any type of motor, such as a stepper motor. By way of example, and without limitation, the motor 601 can be the same type of motor as the motor 301 of the fluid pump 300 discussed above. The motor 601 is coupled to and rotates a rotatable plate 603 between various rotational positions. According to some embodiments, the rotatable plate 603 is coupled to a fixed plate 605 for structural support and rigidity. However, according to some embodiments, the smoking apparatus 600 can omit the fixed plate 605.

In the embodiment with the fixed plate 605, the motor 601 rotates the rotatable plate 603 relative to the fixed plate 605. According to some embodiments, the fixed plate 605 can include an opening that accepts the rotatable plate 603 such that a circumferential outer edge of the rotatable plate 603 engages a circumferential inner edge of the fixed plate 605. Further, as shown in FIG. 6A, the thickness of the rotatable plate 603 can be greater than the thickness of the fixed plate 605 such that the rotatable plate 603 protrudes from the fixed plate 605. Additionally, to support the rotation and maintain the rotatable plate 603 in a fixed position relative to the motor 601, the fixed plate 605 can include one or more bearings 607a (as shown in detail in FIG. 6B) that engage the outer circumferential edge of the rotatable plate 603. Additionally, the smoking apparatus 600 can include a bearing 607b (as shown in detail in FIG. 6D) that engages the outward facing surface of the rotatable plate 603.

To secure a smokeable product 609 within the smoking apparatus 600, the rotatable plate 603 includes a plurality of indents 611, as shown in detail in FIG. 6F. Each indent 611 is configured to engage and secure a smokeable product 609. A smokeable product 609 can include any tobacco-based product that a user (e.g., a human) can smoke, such as, but not limited to, a cigarette, a cigar, a pipe, etc. The smokeable product 609 can also include non-tobacco-based products that a user can smoke, such as a clove cigarette, an electronic cigarette (e-cigarette), etc. Although shown as including 12 indents 611 and 12 secured smokeable products 609, the rotatable plate 603 can be configured (e.g., sized) to include any number of indents 611 to secure any number of smokeable products 609. By way of example, and without limitation, the indents 611 can be specifically designed as cigarette holders and, therefore, be sized to accept and secure a cigarette. However, the indents 611 can be configured (e.g., sized) to accept any type of smokeable product. According to some aspects, the indents 611 can be configured to accept and secure one or more e-cigarettes. Because of the possible additional or greater weight of the e-cigarettes (relative to cigarettes), the indents 611 can be larger and the rotatable plate 603 can be configured to accept and secure the e-cigarettes, such as by an additional mechanical connection to the e-cigarettes beyond merely the indents 611. Such a mechanical connection can include, for example, a latch, a clamp, etc. for each e-cigarette, or for all e-cigarettes as a whole, to connect and secure the e-cigarettes.

According to some embodiments, the rotatable plate 603 can be decoupled from the fixed plate 605 to allow for alternative rotatable plates to be used within the smoking apparatus 600. The alternative rotatable plates can include indents similar to indents 611 that are sized to accept other types of smokeable products, such as cigars rather than cigarettes.

According to some embodiments, the indents 611 of the rotatable plate 603 can be configured to accept multiple types of smokeable products 609. By way of example, and without limitation, an indent 611 can have a step-wise decrease in diameter relative to depth to accept and secure a cigarette and a cigar, such that the cigarette extends and is secured deeper into the indent 611 than the cigar.

As shown in detail in FIG. 6D, each indent 611 includes a throughbore 613 for passing smoke to an opposite side of the rotatable plate 603 from which the smokeable product 609 extends. According to some embodiments, the throughbore 613 is on the rotatable plate 603. Alternatively, in embodiments in which the fixed plate 605 is between the rotatable plate 603 and the motor 601, the throughbore 613 can be within the fixed plate 605. In such an embodiment, the throughbore 613 aligns with an indent 611 for smoking the smokeable product 609 coupled to the indent 611.

As shown in detail in FIGS. 6B-6E, the smoking apparatus 600 further includes a sealing member 615 that includes a seal piece 615a. The sealing member 615 actuates between an engaged position, in which the seal piece 615a engages with and forms a pneumatic seal with the rotatable plate 603 (or fixed plate 605), and a disengaged position, in which the seal piece 615a disengages from the rotatable plate 603 (or fixed plate 605). With the sealing member 615 and the seal piece 615a in the engaged position, the seal piece 615a selectively engages a throughbore 613 of the rotatable plate 603 (or fixed plate 605). With the sealing member 615 and the seal piece 615a in the disengaged position, the rotatable plate 603 can rotate to allow the motor 601 to selectively align a smokeable product 609 and a throughbore 613 with the sealing member 615 and the seal piece 615a. To repeatedly form a pneumatic seal with the rotatable plate 603 (or fixed plate 605), the seal piece 615a can be formed of, for example, polytetrafluoroethylene; however, any elastic material that allows for a pneumatic seal repeatedly made and broken can be used to form the seal piece 615a. The diameter of the seal piece 615a is configured to allow for a pneumatic seal under a minimal amount vacuum. By way of example, and without limitation, the seal piece 615a has a diameter of 1 to 50 mm, such as 6 mm.

The sealing member 615 further includes a plate or holder 617 that is connected to two springs 619. The plate 617 holds the seal piece 615. The springs 619 can be supported around two rods 621. In a relaxed state, the springs 619 apply a compressive force against the plate 617, which causes the sealing member 615 and the seal piece 615a to engage the rotatable plate 603 (or fixed plate 650). In an actuated state, the rods 621 actuate to counteract the compressive force of the springs 619, which causes the seal piece 615a to disengage from the rotatable plate 603 (or fixed plate 605). The actuation force of the rods 621 can be supplied by, for example, the motor 601. Alternatively, the actuation force of the rods 621 can be supplied by an alternative power source, such as a separate motor within a housing 623 (FIG. 6B) that houses the motor 601.

The sealing member 615 further includes an adapter 625 on an opposite side of the plate 617 as the seal piece 615a. The adapter 625 is an interface for tubing, such as tubing 511c, to connect to the sealing member 615. The sealing member 615 includes a channel 627 through the seal piece 615a, the plate 617, and the adapter 625 to permit fluid communication between the throughbore 613 and the tubing 511c. Thus, the tubing 511c allows for the smoke mixed with a fluid (e.g., air) to flow to another component of the system 500, such as the reservoir 503.

By aligning the smokeable product 609 with the sealing member 615 and the tubing exiting from the adapter 625 of the sealing member 615, the resulting arrangement minimizes the deposition of nano-particulates of the one or more agents (e.g., smoke nano-particulates, including tar, etc.) on the associated surfaces, as compared to designs that require bending of the flow of fluid at large angles (e.g., 90° elbow connections) at or adjacent to a connection point between the smokeable product and the tubing of the system.

Although only one sealing member 615 is shown in the smoking apparatus 600, according to some embodiments, the smoking apparatus 600 can include more than one sealing member 615, or the sealing member 615 can include multiple sealing pieces 615a and multiple adapters 625. With multiple sealing members 615, seal pieces 615a, and/or adapters 625, more than one smokeable product 609 can be smoked at once. Further, with multiple sealing members 615, seal pieces 615a, and/or adapters 625, separate tubing can attach separate smokeable products 609 to separate reservoirs 503, as discussed above, from which separate OOCs 10 can sample fluid mixed with the one or more agents. From the reservoirs 503, the tubing can combine into a single piece of tubing leading to the suction pump 501. Thus, the single suction pump 501 can provide the suction needed for each smokeable product 609 in, for example, an experiment comparing the effects of different smokeable products 609 simultaneously.

In some aspects, the rotatable plate 603 and the fixed plate 605 can be omitted from the smoking apparatus 600. Instead, the sealing member 615 can attach directly to the smokeable product 609. For example, the smokeable product 609 can be an e-cigarette. As an e-cigarette, the total duration of smoking the e-cigarette can be greater than, for example, a single, traditional cigarette. Accordingly, the rotatable plate 603 and the fixed plate 605 can be omitted because there is no need to rotate between multiple smokeable products 609. Instead, the single e-cigarette can be used for the entire duration of a test for the effects of whole smoke on the 00C 10. However, as discussed above, the rotatable plate 603 and the fixed plate 605 also can be configured to accept and secure multiple e-cigarettes. In such a case, the effects of, for example, different brands of e-cigarettes and/or different brands or types of aerosols (e.g., e-liquid) can be evaluated during a single test using multiple e-cigarettes.

According to some embodiments, the smoking apparatus 600 includes an ignition system 629. The ignition system 629 automatically ignites one or more of the smokeable products 609. By way of example, and without limitation, the ignition system 629 aligns with a selected smokeable product 609 according to rotation of the rotatable plate 603 under the control of the motor 601. When aligned with the selected smokeable product 609, the ignition system 629 ignites the smokeable product 609.

The ignition system 629 can include various elements for igniting the smokeable product 609, such as a lighter (e.g., open flame), a resistive heating element (e.g., nichrome-wire-based heating element), etc. By way of example, and without limitation, with a nichrome-wire-based heating element, the ignition system 629 can ignite a smokeable product within, for example, 5 seconds. Referring to FIG. 6G, the ignition system 629 includes an ignition coil holder with a conical interior 631 and electromagnetic solenoid 633 for positioning the ignition system 629 against the smokeable product 609. The ignition system 629 can include a conical ignition coil holder to engage the ignition system 629 to a smokeable product. Accordingly, the ignition system 629 allows for the automated ignition of the smokeable product 609 without requiring a user to manually light each smokeable product 609.

The smoking apparatus 600 also includes a controller 635. The controller 635 connects to and controls the motor 601, the sealing piece 615, and the ignition system 629. The controller 635 can be configured according to the controller 327 discussed above. Thus, the controller 635 can operate based on computer instructions, such as processor-executable instructions, for implementing the various functionalities described herein for controlling the motor 601, the sealing member 615, and the ignition system 629. Alternatively, the controller 635 can be a hardware-based controller.

With the sealing member 615, the ignition system 629, and the controller 635, multiple smokeable products can be consecutively smoked by the smoking apparatus 600 to reproduce the effects of a user, for example, smoking multiple cigarettes over time. Further, the automatic rotation of the rotatable plate 603 allows for multiple cigarettes to be inserted within the smoking apparatus 600 and smoked without requiring a user to manually select each separate cigarette. Thus, the smoking apparatus 600 can be set to an automatic mode to expose the 00C 10 to cigarette smoke over a set period of time without requiring a user to continuously and manually control the smoking apparatus 600.

By way of example, and without limitation, the controller 635 causes the motor 601 to selectively align one of the smokeable products 609 with the seal piece 615a of the sealing member 615. The controller 635 then causes the sealing member 615 to engage the seal piece 615a against the rotatable plate 603 (or fixed plate). Then, the controller 635 triggers the ignition system 629 to light the smokeable product 609. With the smokeable product 609 lit, the suction generated by the suction pump 501 draws fluid (e.g., air) through the smokeable product 609 to introduce the smoke into the air. The air mixed with the smoke then travels through the channel 627 of the sealing member and into the tubing 511c. After entering the tubing 511c, the air mixed with the smoke can be directed as desired through the system 500, such as to the reservoir 503 prior to the air and the smoke entering the OOC 10 based on the actuation of the fluid pump 300.

Referring to FIG. 6H, the smoking apparatus 600 can include a cover 637 that surrounds the internals of the smoking apparatus 600. Although shown as opaque, according to some embodiments, the cover 637 is clear to allow an operator to see into the smoking apparatus 600. The cover 637 can be airtight to prevent smoke from the smokeable product from entering, for example, the chamber 509a of the system 500 in FIG. 5 (i.e., when the mixer 505 is the smoking apparatus 600). According to some embodiments, the cover 637 can include one or more ports 639a and 639b. By way of example, and without limitation, the port 639a can connect to tubing 511d to connect to the reservoir 503 for providing second-hand smoke to the reservoir 503 from inside of the cover 637 of the smoking apparatus 600. Further, the port 639b can connect to the exhaust line 515b for allowing smoke within the cover 637 to escape to outside of the chamber 509a (and the system 500). According to some embodiments, a fan (not shown) a can direct fresh air into the smoking apparatus through the exhaust line 515b and the port 639b. Thus, the exhaust line 515b and the port 639b can function both as a fluid inlet and a fluid outlet to allow for fresh air to enter the smoking apparatus 600 through the cover 637 and allow for air mixed with smoke to exit the smoking apparatus through the cover 637. According to some embodiments, the cover 637 can include one or more doors 641 (e.g., sliding doors) to permit access to the smoking apparatus 600.

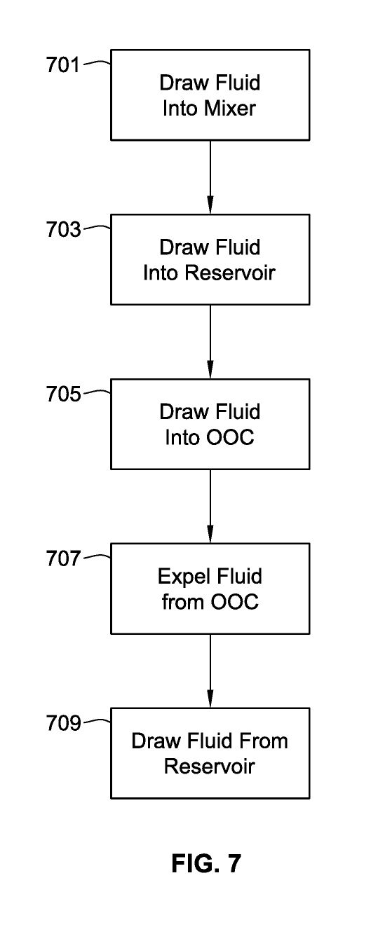

FIG. 7 illustrates one embodiment of a method for exposing the OOC 10 to cigarette smoke by use of the smoking apparatus 600 of FIG. 6A within the system 500 of FIG. 5, in accord with aspects of the present concepts. In this embodiment, the smoking apparatus 600 serves as the mixer 505 and experiments concerning the effects of, for example, cigarette smoke can be conducted on the OOC 10.

Prior to step 701, a user of the system 500 can manually insert one or more cigarettes as the smokeable products 609 into the indents 611 of the rotatable plate 603 of the smoking apparatus 600 of FIG. 6A. The motor 601 of the smoking apparatus 600 rotates, as needed, to align the first cigarette to be smoked with the ignition system 629. However, as discussed above, more than one cigarette can be smoked at once, as needed, based on, for example, the requirements of the desired experiment, the number of OOC 10 in the system 100, etc. The ignition system 629 then operates to ignite the cigarette.

At step 701, the suction pump 501 draws a vacuum within the system 500. As discussed above, depending on the arrangement of the system 500, the vacuum draws fluid into the smoking apparatus 600 (which is the mixer 505 in this embodiment) from the fluid source 507a or the fluid source 507b. For purposes of the following example, the fluid is air; however, the fluid can be any type of fluid for which experiments on the OOC 10 is desired. Further, based on an average smoker's behavior, the average time it takes to smoke a cigarette is 250-300 seconds. Accordingly, the vacuum or suction generated by the suction pump 501 can be controlled to consume a cigarette within about 250 to about 300 seconds.

At step 703, after entering the smoking apparatus 600, the air travels through one of several fluid paths out of the smoking apparatus 600. According to a first path, the air within the smoking apparatus 600 travels through the cigarette under vacuum. In passing through the cigarette, the air mixes with the smoke produced by burning the cigarette, including the various gases and nano-particulates within the smoke. Moreover, because the air passes through the cigarette, the air mixes with smoke directly from the cigarette. The air mixed with the smoke then passes out of the cigarette and through the sealing member 615 of the smoking apparatus and into the tubing 511c that connects to the reservoir 503.

Alternatively, according to a second path, the air within the smoking apparatus 600 mixes with the smoke coming off of the cigarette (e.g., second-hand smoke). Under vacuum, the air mixed with the second-hand smoke exits the smoking apparatus 600 through the tubing 511d that connects to the reservoir 503. Alternatively, according to a third path, the air mixed with the second-hand smoke exits the smoking apparatus 600 through the tubing 511h that connects directly to the tubing 511e at the valve 513c and that leads to the OOC 10, without passing through the reservoir 503.

Whether the air mixed with the smoke takes the first, second, or third path can vary depending whether an experiment using the OOC 10 focuses on first-hand smoke, second-hand smoke, or fresh air. To test the effects of first-hand smoke on the OOC 10, the air mixed with the first-hand smoke from the tubing 511c enters the reservoir 503. To test the effects of second-hand smoke on the OOC 10, the air mixed with the second-hand smoke from the tubing 511d enters the reservoir 503. Alternatively, the air mixed with second-hand smoke from the tubing 511h can bypass the reservoir 503.

At step 705, air mixed with smoke is drawn from the reservoir 503 into the OOC 10 based on the actuation of the fluid pump 300. As discussed above, the motor 301 of the fluid pump 300 rotates the lead screw 303, which causes the traveling nut 317 and the plate 307c to translate about the lead screw 303. To draw air and smoke into the OOC 10, the traveling nut 317 translates about the lead screw 303 to cause the plungers 321 to create a void in the barrels 323. The void created in the barrels 323 creates a vacuum that draws air and smoke from inside of the reservoir 503 into and through the OOC 10. The air and smoke passing through the OOC 10, specifically the microchannel 34 or 36 of the OOC 10, causes a shear stress on the lung cells lining the microchannel 34 or 36, which preferably mimics the shear stress experienced in vivo. Depending on what mixture of fluid and smoke is within the reservoir 503, such as air with first-hand smoke, air with second-hand smoke, or fresh air, the same mixture of air/smoke is drawn into the OOC 10 by the fluid pump 300. Accordingly, the lung cells lining the microchannel 34 or 36 are exposed to the air mixed with the smoke for determining what effects the smoke, in addition to the shear stress, has on the lung cells. Thus, a particulate phase and a gas phase of the smoke are introduced into, for example the microchannel 34 to mimic and test the smoke-induced injury, pathology, toxicity, and carcinogenicity of smoking. According to some embodiments, steps 701, 703, and 705 can occur substantially simultaneously to mimic the action of a smoker taking a puff from a cigarette.

At step 707, the air mixed with smoke is then expelled back through the OOC 10 and back into the reservoir 503 based on the actuation of the fluid pump 300, which provides the bi-directional flow. More specifically, the motor 301 of the fluid pump 300 rotates the lead screw 303, which causes the traveling nut 317 and the plate 307c to translate back about the lead screw 303. Specifically, the traveling nut 317 and the plate 307c translates about the lead screw 303 to cause the plungers 321 to travel back to the original position within the barrels 323. The plungers 321 within the barrels 323 displace the volume of air (or air and smoke) previously within the barrels 323 and expel the air and smoke through the OOC 10 and into the reservoir 503. Again, the air and smoke passing through the OOC 10, specifically the microchannel 34 or 36 of the OOC 10, causes a shear stress on the lung cells lining the microchannel 34 or 36, which mimics the shear stresses experienced in vivo. Accordingly, the lung cells lining the microchannel 34 or 36 are again exposed to the air mixed with the smoke for determining what effects the smoke, in addition to the shear stress, has on the lung cells. At step 709, the air mixed with the smoke within the reservoir 503 is drawn out of the reservoir 503 and expelled out of the exhaust line 515.

Based on an average smoker's behavior for smoking a cigarette, the average volume of air inhaled per puff of a cigarette is about 30 to about 45 ml, such as about 40 ml, with a total volume inhaled through the OOC 10 of, for example, about 150 μl. The average duration of the puff is about 0.9 to about 2 seconds, such as about 1.2 seconds. The average number of puffs per cigarette is about 8 to about 14 puffs, such as 9 puffs, with an average number of breaths per minute of, for example, about 12. The average time between puffs (e.g., inter-puff interval) is about 10 to about 30 seconds, such as about 20 seconds. Accordingly, with respect to steps 705 and 707 discussed above, the motor 301 and the syringes 319 of the fluid pump 300 can be designed and operated so that, for a cigarette burnt during the 250-300 seconds mentioned above, the OOC 10 experiences comparable values of various parameters that the average smoker experiences while smoking a cigarette. By way of example, and without limitation, the motor 301 causes 10 puffs, each lasting about 1.2 seconds and taking in about 150 μl of air mixed with smoke. When multiple cigarettes (or any smokeable product 609) are used, the duration between each cigarette can vary depending on the specifics of the test. For example, the inter-cigarette duration can be as short as the time to rotate between cigarettes. Alternatively, the inter-cigarette duration can be any desired longer duration, such as, for example, about 60 seconds. However, these values can vary depending on determining the effects of, for example, heavy smoking and/or light smoking. Further, a user can control and customize these parameters based on one or more inputs through the input devices 357 of the controller 327 of the fluid pump 300.

According to the arrangement of the smoking apparatus 600 and the fluid pump 300 within the system 500, the generation of smoke is decoupled from the exposure of fluid (e.g., air or air mixed with smoke) within the OOC 10. Accordingly, to further mimic an average smoker smoking a cigarette, puffs from a cigarette that include first-hand cigarette smoke can occur between periods during which the OOC 10 are exposed to fresh air within the chamber 509a by actuation of a valve in the tubing 511e (or air mixed with second-hand smoke from a second reservoir). Accordingly, steps 705 and 707 can be repeated in which fresh air is drawn into the reservoir 503 and the OOC 10, which replaces the air mixed with smoke.

By way of example, and without limitation, the suction pump 501 can draw out all of the air mixed with smoke from the reservoir 503 and replace it with fresh air from the inlet port 517. Accordingly, repeated actuation of the fluid pump 300 with fresh air within the reservoir 503 mimics breaths of fresh air a smoker may take between puffs from a cigarette. When ready to repeat steps 705 and 707 with first-hand smoke, the reservoir 503 is again filled with air mixed with first-hand cigarette smoke, as discussed above with respect to steps 701 and 703, by the vacuum created by the suction pump 501 taking a puff from the cigarette. Such control is based, at least in part, on the valves 513a-513d within the system 500 and directionality of flow based on the suction pump 501 and the fluid pump 300. Accordingly, the system can mimic breathing smoke in, exhaling the smoke out, and then breathing fresh air and fresh air out (e.g., inter-puff interval), and so on, which more precisely mimics the flow of air and smoke flow in and out of the lungs of smokers.

Alternatively, by way of another example, and without limitation, the suction pump 501 may draw out all of the air mixed with first-hand smoke from the reservoir 503 and replace it with air mixed with second-hand smoke from the smoking apparatus 600 through the tubing 511d. Accordingly, repeated actuation of the fluid pump 300 with air mixed with second-hand smoke within the reservoir 503 mimics breaths of air mixed with second-hand smoke from the cigarette that a smoker may take between puffs from the cigarette. When ready to repeat steps 705 and 707 with first-hand cigarette smoke, the reservoir 503 is again filled with air mixed with first-hand cigarette smoke, as discussed above with respect to steps 701 and 703.

To achieve the process steps of cycling different air within the reservoir 503 and synchronizing the suction pump 501 and the fluid pump 300 to simulate a smoker taking a puff of smoke from a cigarette, the fluid pump 300 and the suction pump 501 are synchronized so that the operation of the fluid pump 300 draws in the appropriate amount of air with first- or second-hand smoke (or fresh air). Accordingly, the controller 327 of the fluid pump 300 is in communication with the controller 635 of the smoking apparatus 600. Alternatively, according to some embodiments, a single controller (e.g., controller 327 or 635) can control both the fluid pump 300 and the smoking apparatus 600 to ensure synchronized operation. One or both of the controllers 327 and 635 also can control the valves 513a-513d for directing the fluid mixed with the one or more agents through the system 500.

According to the method illustrated and described with respect to FIG. 7, by drawing the smoke (e.g., one or more agents) into the OOC 10 using the fluid pump 300, the cells lining the microchannel 34 or 36 within the OOC 10 mimic a smoker's lungs during smoking to model lung pathologies and to be able to model smoke-triggered airway diseases like Chronic Obstructive Pulmonary Disease (COPD). This same approach can be taken to study the effects of other types of smoke and associated particulates (e.g., as in produced by burning of clothing, construction materials, household chemical and materials, or any other material) on lung pathophysiology by replacing the smoking apparatus 600 with another type of mixer 505.

According to the arrangement illustrated and described with respect to FIG. 5 and the method of FIG. 7, the fluid pump 300 can draw in physiologically relevant volumes of smoke within the OOC 10 over the course of an experiment in a manner consistent with the way individuals tend to smoke. Having this control is especially important when considering the relative air volume, velocity, and pause between smoking in and out. For the OOC 10 compared to human lungs, only a small amount of the total smoke generated by the smoking apparatus 600 is needed for proper exposure levels. The decoupling of smoke generation from smoke sampling enables not only physiologically relevant smoke exposure levels but also temporal sampling that closely matches the way individuals smoke. As described above, the smoking apparatus 600 can be replaced with other machines that introduce other agents (e.g., chemicals and/or particulates associated with the burning of any material, engine exhaust fumes, etc.) to adapt the system 500 to explore the effects of smoke or airborne components produced by other sources.

For certain types of agents within the fluid, such as cigarette smoke, and depending on the volumes of fluid mixed with the one or more agents being drawn into the OOC 10, the volume in the tubing 511e can become significant. If the volume in the tubing 511e is greater than the volume being drawn into the OOC 10, the OOC 10 is not exposed to the one or more agents within the fluid. Rather, most of the one or more agents are pulled through the reservoir 503 into the suction pump 501. Therefore, the sizing, location, and placement of the components of the system 500 are designed to minimize the amount of dead volume in the tubing 511e between the reservoir 503 and the OOC 10. By way of example, and without limitation, the reservoir 503 is placed in close proximity to the OOC 10. Additionally, for the tubing 511e leading to the OOC 10, the tubing 511e has an internal diameter of 250 μm to reduce the amount of dead volume. However, this dimension can be altered to match any system design modifications or improvements. By way of example, and without limitation, when larger volumes of fluid are desired for delivery to the OOC 10, tubing used as the tubing 511e can have a larger diameter to create less resistance to fluid flow. At the same time, the length of the tubing 511e can be short to allow for an acceptable amount of dead volume (e.g., as a fraction of the total fluid delivered to the OOC 10). According to some embodiments, larger tubing with still acceptable amounts of dead volume can be used as the tubing 511e to allow for easier handling of, for example, the connection and the disconnection of the tubing 511e within the system 500. Further, for other tubing throughout the system 500, larger diameter tubing can be used to reduce the effects of clogging resulting from, for example, nano-particulates being introduced into the fluid within the system 500. For example, tubing with a larger diameter than 250 μm can be used elsewhere than tubing 511e throughout the system 500. Further, the tubing used within the system 500 can be formed of various materials, such as polyurethane, and can be disposed of between different experiments, or different and dissimilar experiments.

Based on the system 500, and as modified with respect to, for example, the mixer 505 that allows for the introduction of various agents into various different fluids that are then exposed to the OOC 10, the system allows for various different applications to test the effects of fluid and/or agents on the OOC 10. By way of example, and without limitation, such applications include analyzing the effects of patho-physiologically relevant airflow shear on top of lung epithelial cells and any other cell types in the OOC 10 (or other culture devices containing micro- or meso-scale fluidic channels), studying the mechanical stress of delivering air and liquids using this system in lung/airway chips as well as other organ chips, and mimicking different breathing patterns (e.g., deep sigh, exercise, resting, coughing, etc.).

As discussed in detail above, the system 500 with the smoking apparatus 600 or another type of mixer 505 (e.g., pressurized supply of fluid and/or one or more agents) allows for the exposure one or more OOC 10 to cigarette smoke and/or other tobacco-related products (e.g., cigar, hookah, e-cigarette, etc.) and/or aerosolized particles to mimic a smoker's lungs and study biochemical changes associated with such exposure.

Based on the ability to include multiple cigarettes within the smoking apparatus 600 at once, the system 500 with the smoking apparatus also allows for the comparison of various tobacco-related products, such as according to type (e.g., cigar versus cigarette) or to brand (e.g., different brands of cigarette), for their cytotoxicity, metabolism by epithelial cells, cellular stress, and inflammation induction, as examples.

The ability to modify the mixer 505 within the system 500 affords the opportunity to test various different effects on the lung cells, such as, but not limited to, studying the effects of smoke produced by burning of any substance (e.g., clothes, building materials, car upholstery, etc.), fumes in air (e.g., engine exhaust, bomb explosion fall out, etc.), aerosolized drugs, particulates, toxins, etc. Modification of the system 500 to omit the mixer 505, or to have the fluid pump 300 in fluid communication with the OOC 10 and a fluid source also allows for testing the various different effects on the lung cells that are unrelated to one or more agents within the fluid, such as, but not limited to, studying ventilation-induced lung injury by modeling airway closure and opening due to repeated passage of an air-liquid interface atop epithelial cells.

According to some embodiments, although the system 500 has been described primarily with respect to the fluid pump 300 drawing fluid into and out of the OOC 10, according to some embodiments the fluid pump 300 can be replaced with a mechanical actuator, a pneumatic actuator, or a pump, depending on the requirements of the system 500.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

1. A microfluidic system for determining a response of cells, comprising:
one or more fluid pumps for moving a fluid across cells within a microfluidic device, the microfluidic device including a microchannel at least partially defined by a surface having cells adhered thereto, a first port at one end of the microchannel, and a second port at an opposing end of the microchannel,
wherein the one or more fluid pumps move the fluid across the cells in a first direction toward the second port and then move the fluid across the cells in a second direction toward the first port.

2. The microfluidic system of claim 1, wherein the cells are airway cells and the moving of the fluid in the first direction and the second direction is for simulating a movement of air associated with a respiratory function.

3. The microfluidic system of claim 2, further comprising:
a controller electrically connected to the one or more fluid pumps for controlling the one or more fluid pumps according to a control profile to provide a volume of the fluid across the airway cells, a velocity of the fluid across the airway cells, a cyclical pattern of the fluid across the airway cells, or a combination thereof that mimics the respiratory function.

4. The microfluidic system of claim 1, further comprising:
an agent introduction apparatus in fluid communication with the microfluidic device through the first port for introducing one or more agents into the fluid.

5. The microfluidic system of claim 4, wherein the agent introduction apparatus is a smoking apparatus.

6. The microfluidic system of claim 5, wherein the one or more agents comprise cigarette smoke, and the response of the cells is based on the cigarette smoke.

7. The microfluidic system of claim 4, further comprising:
a reservoir between and in fluid communication with the microfluidic device and the agent introduction apparatus along a first path for holding the fluid mixed with the one or more agents prior to the fluid and the one or more agents moving across the cells in the first direction.

8. The microfluidic system of claim 7, further comprising:
a fluid-connection between and in fluid communication with the microfluidic device and the agent introduction apparatus forming a second path for introducing the fluid containing the one or more agents into the microfluidic device, bypassing the reservoir.

9. The microfluidic system of claim 4, further comprising:
a suction pump in fluid communication with the agent introduction apparatus for drawing the fluid containing the one or more agents from the agent introduction apparatus.

10. The microfluidic system of claim 1, further comprising:
an exhaust port, wherein the system is adapted to selectively expel the fluid through the exhaust port.

11. The microfluidic system of claim 1, further comprising:
a chamber housing the microfluidic device and the one or more fluid pumps at a controlled temperature.

12. The microfluidic system of claim 1, wherein the one or more fluid pumps are selected from syringe pumps, peristaltic pumps, compressor pumps, diaphragm pumps, and piston pumps.

13. The microfluidic system of claim 1, wherein at least one pump of the one or more pumps includes:
at least one syringe including a movable plunger and having an end with a port in fluid communication with the microfluidic device;
a traveling nut coupled to the at least one syringe;
a motor coupled to the traveling nut for moving the traveling nut in one direction to actuate the at least one syringe resulting in the fluid moving into the port and for moving the traveling nut in another direction to actuate the at least one syringe resulting in the fluid moving out of the port; and
a controller configured to control the motor according to a control profile to provide a volume, a velocity, a cyclical pattern, or a combination thereof of the fluid within the microfluidic device that mimics a respiratory function.

14. A fluid pump for producing bi-directional movement of a fluid within one or more microfluidic devices, comprising:
at least one syringe comprising a movable plunger and having an end with a port in fluid communication with at least one of the microfluidic devices;
a traveling nut coupled to the at least one syringe; and
a motor coupled to the traveling nut for moving the traveling nut in a first direction to actuate the at least one syringe resulting in the fluid moving into the port and for moving the traveling nut in a second direction to actuate the at least one syringe resulting in the fluid moving out of the port.

15. The fluid pump of claim 14, further comprising:
a controller configured to control the motor according to a control profile to provide a volume, a velocity, a cyclical pattern, or a combination thereof of the fluid within the one or more microfluidic devices that mimics a respiratory function.

16. The fluid pump of claim 14, the at least one syringe comprising a barrel that supports the moveable plunger and includes the port, the fluid pump further comprising:
a first plate fixed to the barrel at the end of the at least one syringe;
a second plate fixed to the moveable plunger;
a lead screw extending between the first plate and the second plate, wherein the traveling nut is coupled to one of the first plate and the second plate and translates about the lead screw based on a rotation of the motor; and
a guide rail supported by the first plate and the second plate to restrict movement of the one of the first plate and the second plate to the translation about the lead screw.

17. The fluid pump of claim 16, wherein the first plate comprises a gasket, the gasket coupling the barrel to the first plate and to a tube that connects to the port.

18. The fluid pump of claim 14, wherein the motor is a stepper motor and a volume of the fluid moved within the one or more microfluidic devices is controlled based on a number of rotation steps of the stepper motor.

19. The fluid pump of claim 14, further comprising:
a limit switch for triggering a home position of the traveling nut between cyclical operations of the motor.

20. A microfluidic device for determining a response of cells, comprising:
a body that at least partially defines a first microchannel and a second microchannel, the first microchannel for bi-directionally flowing fluid through the microfluidic device and the second microchannel for flowing fluid through the microfluidic device; and
a porous membrane that at least partially defines the first microchannel and the second microchannel, the porous membrane includes the cells on at least a portion thereof that partially defines the first microchannel.

21. An apparatus for introducing smoke into a fluid for delivery to a microfluidic device, comprising:
a plate comprising one or more indents on a first side, each indent configured to couple a smokeable product to the plate, wherein the one or more smokeable products extend from the plate in a first direction;
a seal piece configured to selectively engage with the plate to create a seal on a second side of the plate, opposing one of the one or more indents; and
a tube coupled to and extending from the seal piece in a second direction opposite to the first direction,
wherein the tube is in fluid communication with the microfluidic device for supplying the fluid and the smoke to the microfluidic device.

22. The apparatus of claim 21, wherein the plate includes a plurality of the indents, and the apparatus further comprising:
a motor coupled to the plate for rotating the plate to selectively align the one of the plurality of smokeable products with the seal piece.

23. The apparatus of claim 22, further comprising:
a frame coupled to the seal piece for translating the seal piece between a first position engaged with the plate and a second position disengaged from the plate,
wherein the translation of the frame coupled to the seal piece is synchronized with the rotation of the plate by the motor to disengage the seal piece from the plate prior to rotating the plate and to engage the seal piece with the plate after rotating the plate.

24. The apparatus of claim 21, further comprising:
an ignition system to selectively ignite the one or more coupled smokeable products to cause the one or more smokeable products to generate the smoke.

25. A method of bi-directionally flowing a gaseous mixture comprising air mixed with smoke, comprising:
a) providing i) a microfluidic device comprising a body that at least partially defines a microchannel, and ii) the gaseous mixture;
b) introducing a portion of said gaseous mixture into said microchannel so as to cause said gaseous mixture to move in a first direction; and
c) causing said gaseous mixture to move in a second direction, thereby bi-directionally flowing said gaseous mixture.

26. The method of claim 25, wherein said microchannel comprises a membrane, and said microchannel comprises cells.

27. The method of claim 25, wherein said microchannel comprises cells.

28. The method of claim 27, wherein said microfluidic device further comprises a first port at one end of the microchannel, and a second port at an opposing end of the microchannel,
wherein the gaseous mixture in step b) moves across the cells in a first direction toward the second port and the gaseous mixture in step c) moves across the cells in a second direction toward the first port.

29. The method of claim 28, wherein said gaseous mixture is caused to move via a pump.

30. The method of claim 28, wherein said gaseous mixture is provided in a reservoir.

31. A method for introducing smoke to a microfluidic device, comprising:
a) providing a microfluidic device in fluid communication with a smoking device, said smoking device comprising i) a receptacle with a smokeable product coupled thereto, said smokeable product capable of generating smoke when ignited, and ii) a tube in fluid communication with said microfluidic device for supplying the smoke to the microfluidic device, said microfluidic device comprising a body that at least partially defines a microchannel, said body comprising cells;
b) igniting said smokeable product under conditions that generate smoke;
c) mixing said smoke with air to create a mixture; and
d) delivering said air and smoke mixture to said microchannel under conditions such that said mixture contacts said cells.

32. The method of claim 31, wherein said receptacle comprises a plate with indents, each indent configured to couple a smokeable product to said plate.

33. The method of claim 31, wherein said smokeable product comprises a cigarette.

34. The method of claim 30, wherein the concentration of smoke in said reservoir is consistent with first-hand smoke.

35. The method of claim 30, wherein the concentration of smoke in said reservoir is consistent with second-hand smoke.

36. The method of claim 31, wherein a concentration of smoke in said mixture is consistent with first-hand smoke.

37. The method of claim 31, wherein a concentration of smoke in said mixture is consistent with second-hand smoke.

38. A method of stimulating endothelial cells, comprising:
a) providing i) a microfluidic device comprising a body, said body comprising endothelial cells and ii) a cigarette smoke extract; and
b) introducing a portion of said cigarette smoke extract into said microfluidic device so as to stimulate said endothelial cells.

39. The method of claim 38, wherein said extract is contained in culture media.

40. The method of claim 39, wherein said culture media flows over said cells in a first direction, and then moves in a second direction.

41. The method of claim 38, wherein said body further comprises a membrane.
